SEQUENCE LISTING (41 sequences)

SEQ ID NO: 1 - protein, 330 aa, Homo sapiens [sequence omitted]

SEQ ID NO: 2 - protein, 98 aa, Artificial Sequence
  CH1 IgG1 (positions 1-98 of P01857-1, v1)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 3 - protein, 12 aa, Artificial Sequence
  Hinge IgG1 (positions 99-110 of P01857-1, v1)
EPKSCDKTHTCP

SEQ ID NO: 4 - protein, 113 aa, Artificial Sequence
  CH2 IgG1 (positions 111-223 of P01857-1, v1)
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 5 - protein, 107 aa, Artificial Sequence
  CH3 IgG1 (positions 224-330 of P01857-1, v1)
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 220 aa, Artificial Sequence
  CH2-CH3 IgG1
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 107 aa, Artificial Sequence [sequence omitted]

SEQ ID NO: 8 - protein, 113 aa, Artificial Sequence
  CH2_GASD
PCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 9 - protein, 113 aa, Artificial Sequence
  CH2_GASDIE
PCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAK

SEQ ID NO: 10 - protein, 113 aa, Artificial Sequence
  CH2_GASDALIE
PCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAK

SEQ ID NO: 11 - protein, 113 aa, Artificial Sequence
  CH2_LCKC
PCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAK

SEQ ID NO: 12 - protein, 113 aa, Artificial Sequence
  CH2_GASDLCKC
PCPAPELLAGPDVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAK

SEQ ID NO: 13 - protein, 113 aa, Artificial Sequence
  CH2_GASDIELCKC
PCPAPELLAGPDVFCFPPKPKDTLMISRTPEVTCVVVDVSHEDPEVKFNWYVDGVEVHNAKTKPREEQYNSTYRVVSVLTVLHQDWLNGKEYKCKVSNKALPAPEECTISKAK

SEQ ID NO: 14 - protein, 113 aa, Artificial Sequence
  CH2_GASDALIELCKC
PCPAPELLAGPDVFCFPPKPKDTLMISRTPEVTCVVVDVSHEDPEVKFNWYVDGVEVHNAKTKPREEQYNSTYRVVSVLTVLHQDWLNGKEYKCKVSNKALPLPEECTISKAK

SEQ ID NO: 15 - protein, 107 aa, Artificial Sequence
  CH3_EKEG
GQPRKPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 16 - protein, 220 aa, Artificial Sequence
  CH2-CH3_GASD
PCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 220 aa, Artificial Sequence
  CH2-CH3_GASDIE
PCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 220 aa, Artificial Sequence
  CH2-CH3_GASDALIE
PCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 220 aa, Artificial Sequence
  CH2-CH3_LCKC
PCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 220 aa, Artificial Sequence
  CH2-CH3_GASDLCKC
PCPAPELLAGPDVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 220 aa, Artificial Sequence
  CH2-CH3_GASDIELCKC
PCPAPELLAGPDVFCFPPKPKDTLMISRTPEVTCVVVDVSHEDPEVKFNWYVDGVEVHNAKTKPREEQYNSTYRVVSVLTVLHQDWLNGKEYKCKVSNKALPAPEECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 220 aa, Artificial Sequence
  CH2-CH3_GASDALIELCKC
PCPAPELLAGPDVFCFPPKPKDTLMISRTPEVTCVVVDVSHEDPEVKFNWYVDGVEVHNAKTKPREEQYNSTYRVVSVLTVLHQDWLNGKEYKCKVSNKALPLPEECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 220 aa, Artificial Sequence
  CH2-CH3_EKEG
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 24 - protein, 220 aa, Artificial Sequence
  CH2-CH3_LCKCEKEG
PCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPRKPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 25 - protein, 330 aa, Artificial Sequence
  Mouse Ig gamma-2A chain C region, A allele (UniProtP01863-1, v1)
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 26 - protein, 5 aa, Artificial Sequence
  G4S linker
GGGGS

SEQ ID NO: 27 - protein, 15 aa, Artificial Sequence
  (G4S)3 linker
GGGGSGGGGSGGGGS

SEQ ID NO: 28 - protein, 330 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 98 aa, Artificial Sequence
  CH1 IgG1 (G1m3)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 30 - protein, 107 aa, Artificial Sequence
  CH3 IgG1 (G1m3)
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 220 aa, Artificial Sequence
  CH2-CH3 (G1m3)
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 107 aa, Artificial Sequence
  CH3_EKEG (G1m3)
GQPRKPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 33 - protein, 220 aa, Artificial Sequence
  CH2-CH3_GASD (G1m3)
PCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 220 aa, Artificial Sequence
  CH2-CH3_GASDIE (G1m3)
PCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 220 aa, Artificial Sequence
  CH2-CH3_GASDALIE (G1m3)
PCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 220 aa, Artificial Sequence
  CH2-CH3_LCKC (G1m3)
PCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 220 aa, Artificial Sequence
  CH2-CH3_GASDLCKC (G1m3)
PCPAPELLAGPDVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 220 aa, Artificial Sequence
  CH2-CH3_GASDIELCKC (G1m3)
PCPAPELLAGPDVFCFPPKPKDTLMISRTPEVTCVVVDVSHEDPEVKFNWYVDGVEVHNAKTKPREEQYNSTYRVVSVLTVLHQDWLNGKEYKCKVSNKALPAPEECTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 220 aa, Artificial Sequence
  CH2-CH3_GASDALIELCKC (G1m3)
PCPAPELLAGPDVFCFPPKPKDTLMISRTPEVTCVVVDVSHEDPEVKFNWYVDGVEVHNAKTKPREEQYNSTYRVVSVLTVLHQDWLNGKEYKCKVSNKALPLPEECTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 220 aa, Artificial Sequence
  CH2-CH3_EKEG (G1m3)
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 41 - protein, 220 aa, Artificial Sequence
  CH2-CH3_LCKCEKEG (G1m3)
PCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPRKPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

(19) United States
(12) Patent Application Publication
(10) Pub. No.: US 2021/0388083 A1
(43) Pub. Date: Dec. 16, 2021

(54) ENGINEERED FC

(71) Applicant: Hummingbird Bioscience Holdings Pte. Ltd., Singapore (SG)

(72) Inventors: Jerome Douglas Boyd-Kirkup, Singapore (SG); Piers Ingram, Singapore (SG); Vicente Sancenon, Singapore (SG)

(73) Assignee: Hummingbird Bioscience Holdings Pte. Ltd., Singapore (SG)

(21) Appl. No.: 17/288,245

(22) PCT Filed: Oct. 25, 2019
(86) PCT No.: PCT/EP2019/079131

§ 371 Date: Apr. 23, 2021

(30) Foreign Application Priority Data
Oct. 25, 2018 (GB) 1817354.2

(51) Int. Cl. C07K 16/28; C07K 16/32

(52) U.S. Cl. CPC C07K 16/283; C07K 16/32; C07K 2317/72; C07K 2317/526; C07K 2317/732; C07K 2317/524

(57) ABSTRACT

Antigen-binding molecules comprising an Fc region comprising a polypeptide having: (i) C at the position corresponding to position 242, and C at the position corresponding to position 334, and (ii) one or more of: A at the position corresponding to position 236, D at the position corresponding to position 239, E at the position corresponding to position 332, L at the position corresponding to position 330, K at the position corresponding to position 345, and G at the position corresponding to position 430 are disclosed. Also discloses are constituent polypeptides of such Fc regions, nucleic acids encoding such antigen- binding molecules and polypeptides, compositions comprising such antigen-binding molecules, polypeptides and nucleic acids, and methods using the same.

This application claims priority from GB 1817354.2 filed 25 Oct. 2018, the contents and elements of which are herein incorporated by reference for all purposes.

### FIELD OF THE INVENTION

The present invention relates to the fields of molecular biology, more specifically antibody technology.

### BACKGROUND TO THE INVENTION

There are two main (non-mutually exclusive) strategies to modulate (enhance or attenuate) antibody Fc effector function (ADCC, ADCP, CDC) by altering Fc:Fc receptor and Fc:complement component 1q (C1q) interactions.

The most common approach involves providing amino acid substitutions to the polypeptide chains of the Fc region to create symmetric (homodimeric) IgG molecules.

Alternatively, antibodies can be glycoengineered; the most common strategies include modification of N-linked oligosaccharides by manipulating glycan biosynthetic pathways in host cells, and in vitro remodelling of glycans. Modifications include defucosylation, increased terminal galactosylation and increased terminal sialylation.

However, there are drawbacks associated with known modifications to influence effector function. Afucosyl antibodies are technically challenging to produce, typically requiring expression in mutant cell types (e.g. Lec13 CHO cells, FUT8 knockout CHO cells etc.) or expression from cells treated to reduce expression of factors involved in glycan synthesis/processing (e.g. cells treated with FUT8 siRNA or kifunensine inhibitors), or requiring treatment of antibody preparations after their exoression to remove glycans having fucosyl residues. Such antibody preparations are often contaminated by fucosylated antibody, such that the improvement in Fcγ receptor binding activity over fucosylated antibody preparations is limited to ˜3 times (see e.g. Chung et al., MAbs (2012) 4(3): 326-340).

Contemporary approaches to improving effector function through the introduction of amino acid substitutions in the Fc region are generally associated with ˜2-5 times improvement in ADCC activity relative to antibodies having an unsubstituted Fc region.

There remains a need for Fc regions with improved structural and functional properties.

### SUMMARY OF THE INVENTION

In a first aspect the present invention provides an antigen-binding molecule, optionally isolated, comprising an Fc region, the Fc region comprising a polypeptide having: (i) C at the position corresponding to position 242, and C at the position corresponding to position 334, and (ii) one or more of: A at the position corresponding to position 236, D at the position corresponding to position 239, E at the position corresponding to position 332, L at the position corresponding to position 330, K at the position corresponding to position 345, and G at the position corresponding to position 430.

Unless stated otherwise, positions in polypeptides of Fc regions are numbered according to the EU numbering system as described in Kabat et al., Sequences of Proteins of Immunological Interest, 5th Ed. Public Health Service, National Institutes of Health, Bethesda, Md., 1991.

In some embodiments, the Fc region comprises a polypeptide having: (i) C at the position corresponding to position 242, and C at the position corresponding to position 334, and (ii) A at the position corresponding to position 236, D at the position corresponding to position 239, E at the position corresponding to position 332, and L at the position corresponding to position 330; or A at the position corresponding to position 236, D at the position corresponding to position 239, and E at the position corresponding to position 332; or A at the position corresponding to position 236, and D at the position corresponding to position 239; or K at the position corresponding to position 345, and G at the position corresponding to position 430.

In some embodiments, the Fc region comprises a polypeptide having: (i) C at the position corresponding to position 242, and C at the position corresponding to position 334, and (ii) A at the position corresponding to position 236, D at the position corresponding to position 239, E at the position corresponding to position 332, and L at the position corresponding to position 330.

In some embodiments, the Fc region comprises a polypeptide having: (i) C at the position corresponding to position 242, and C at the position corresponding to position 334, and (ii) A at the position corresponding to position 236, D at the position corresponding to position 239, and E at the position corresponding to position 332.

In some embodiments, the Fc region comprises a polypeptide having: (i) C at the position corresponding to position 242, and C at the position corresponding to position 334, and (ii) A at the position corresponding to position 236, and D at the position corresponding to position 239.

In some embodiments, the Fc region comprises a polypeptide having: (i) C at the position corresponding to position 242, and C at the position corresponding to position 334, and (ii) K at the position corresponding to position 345, and G at the position corresponding to position 430.

In some embodiments, the Fc region comprises a polypeptide comprising an amino acid sequence having at least 60% sequence identity to SEQ ID NO:39, 38, 37, 41, 22, 21, 20 or 24.

Also provided is a polypeptide, optionally isolated, comprising: an amino acid sequence having at least 60% sequence identity to SEQ ID NO:31 or 6, wherein the polypeptide comprises the following amino acid residues at the specified positions numbered relative to SEQ ID NO:31 or 6: (i) C at position 15, and C at position 107, and (ii) one or more of: A at position 9, D at position 12, L at position 103, E at position 105, K at position 118, and G at position 203.

In some embodiments, the polypeptide comprises the following amino acid residues at the specified positions numbered relative to SEQ ID NO:31 or 6: (i) Cat position 15, and C at position 107, and (ii) A at position 9, D at position 12, L at position 103, and E at position 105; or A at position 9, D at position 12, and E at position 105; or A at position 9, and D at position 12; or K at position 118, and G at position 203.

In some embodiments, the polypeptide comprises the following amino acid residues at the specified positions numbered relative to SEQ ID NO:31 or 6: (i) C at position 15, and C at position 107, and (ii) A at position 9, D at position 12, L at position 103, and E at position 105.

In some embodiments, the polypeptide comprises the following amino acid residues at the specified positions numbered relative to SEQ ID NO:31 or 6: (i) C at position 15, and C at position 107, and (ii) A at position 9, D at position 12, and E at position 105.

In some embodiments, the polypeptide comprises the following amino acid residues at the specified positions numbered relative to SEQ ID NO:31 or 6: (i) C at position 15, and C at position 107, and (ii) A at position 9, and D at position 12.

In some embodiments, the polypeptide comprises the following amino acid residues at the specified positions numbered relative to SEQ ID NO:31 or 6: (i) C at position 15, and C at position 107, and (ii) K at position 118, and G at position 203.

Also provided is a polypeptide, optionally isolated, comprising the amino acid sequence of SEQ ID NO:39, 38, 37, 41, 22, 21, 20 or 24.

Also provided is an Fc region, optionally isolated, comprising a polypeptide as described herein. Also provided is an antigen-binding molecule, optionally isolated, comprising a polypeptide or Fc region as described herein.

Also provided is a nucleic acid, or a plurality of nucleic acids, optionally isolated, encoding an antigen-binding molecule, polypeptide or Fc region as described herein.

Also provided is an expression vector, or a plurality of expression vectors, comprising a nucleic acid or a plurality of nucleic acids as described herein.

Also provided is a cell comprising an antigen-binding molecule, polypeptide, Fc region, a nucleic acid or plurality of nucleic acids, or expression vector or plurality of expression vectors as described herein.

Also provided is a method comprising culturing a cell comprising a nucleic acid or a plurality of nucleic acids or expression vector or a plurality of expression vectors as described herein under conditions suitable for expression of the antigen-binding molecule, polypeptide or Fc region from the nucleic acid(s) or expression vector(s).

Also provided is a composition comprising an antigen-binding molecule, polypeptide, Fc region, a nucleic acid or plurality of nucleic acids, expression vector or plurality of expression vectors or a cell as described herein.

Also provided is an antigen-binding molecule, polypeptide, Fc region, a nucleic acid or plurality of nucleic acids, expression vector or plurality of expression vectors, cell, or composition as described herein for use in a method of medical treatment or prophylaxis.

Also provided is an antigen-binding molecule, polypeptide, Fc region, a nucleic acid or plurality of nucleic acids, expression vector or plurality of expression vectors, cell, or composition as described herein for use in a method of treatment or prevention of a cancer, an infectious disease or an autoimmune disease.

Also provided is the use of an antigen-binding molecule, polypeptide, Fc region, a nucleic acid or plurality of nucleic acids, expression vector or plurality of expression vectors, cell, or composition as described herein in the manufacture of a medicament for use in a method of treatment or prevention of a cancer, an infectious disease or an autoimmune disease.

Also provided is a method of treating or preventing a cancer, an infectious disease or an autoimmune disease, comprising administering to a subject a therapeutically or prophylactically effective amount of an antigen-binding molecule, polypeptide, Fc region, a nucleic acid or plurality of nucleic acids, expression vector or plurality of expression vectors, cell, or composition as described herein.

Also provided is a method, optionally an in vitro method, of killing cells expressing a target antigen, comprising contacting cells expressing the target antigen with an antigen-binding molecule, polypeptide, Fc region, a nucleic acid or plurality of nucleic acids, expression vector or plurality of expression vectors, cell, or composition as described herein.

Description

The present invention is based on the unexpected finding that particular combinations of Fc region substitutions are useful for providing antigen-binding molecules with combinations of advantageous properties.

In particular, the inventors have identified that Fc region substitutions for providing an intramolecular disulphide bridge can be combined with Fc region substitutions for enhancing effector activity, to arrive at Fc regions possessing unexpectedly high affinity for activatory Fcγ receptors and FcRn receptors, high selectivity for activatory Fcγ receptors over inhibitory Fcγ receptors, and high stability.

Antigen-Binding Molecules

The present invention provides antigen-binding molecules. The antigen-binding molecules may be provided in isolated or substantially purified form.

An “antigen-binding molecule” refers to a molecule which is capable of binding to a target antigen, and encompasses monoclonal antibodies, polyclonal antibodies, monospecific and multispecific antibodies (e.g., bispecific antibodies), and antibody fragments (e.g. Fv, scFv, Fab, scFab, F(ab′)₂, Fab₂, diabodies, triabodies, scFv-Fc, minibodies, single domain antibodies (e.g. VhH), etc.), as long as they display binding to the relevant target molecule(s).

The antigen-binding molecule of the present invention comprises a moiety capable of binding to a target antigen, and an Fc region.

In some embodiments, the moiety capable of binding to a target antigen comprises an antibody heavy chain variable region (VH) and an antibody light chain variable region (VL) of an antibody capable of specific binding to the target antigen.

In some embodiments, the moiety capable of binding to a target antigen comprises or consists of an aptamer capable of binding to the target antigen, e.g. a nucleic acid aptamer (reviewed, for example, in Zhou and Rossi Nat Rev Drug Discov. 2017 16(3):181-202). In some embodiments, the moiety capable of binding to a target antigen comprises or consists of a antigen-binding peptide/polypeptide, e.g. a peptide aptamer, thioredoxin, monobody, anticalin, Kunitz domain, avimer, knottin, fynomer, atrimer, DARPin, affibody, nanobody (i.e. a single-domain antibody (sdAb)) affilin, armadillo repeat protein (ArmRP), OBody or fibronectin—reviewed e.g. in Reverdatto et al., Curr Top Med Chem. 2015; 15(12): 1082-1101, which is hereby incorporated by reference in its entirety (see also e.g. Boersma et al., J Biol Chem (2011) 286:41273-85 and Emanuel et al., Mabs (2011) 3:38-48).

The antigen-binding molecules of the present invention generally comprise an antigen-binding moiety comprising a VH and a VL of an antibody capable of specific binding to the target antigen. The antigen-binding moiety formed by a VH and a VL may also be referred to herein as an Fv region.

An antigen-binding molecule may be, or may comprise, an antigen-binding polypeptide, or an antigen-binding polypeptide complex. An antigen-binding molecule may comprise more than one polypeptide which together form an antigen-binding domain. The polypeptides may associate covalently or non-covalently. An antigen-binding molecule may refer to a non-covalent or covalent complex of more than one polypeptide (e.g. 2, 3, 4, 6, or 8 polypeptides), e.g. an IgG-like antigen-binding molecule comprising two heavy chain polypeptides and two light chain polypeptides.

The antigen-binding molecules of the present invention may be designed and prepared using the sequences of monoclonal antibodies (mAbs) capable of binding to a target antigen. An antigen-binding moiety of an antibody is any fragment of an antibody which is capable of binding to the target for which the given antibody is specific. Antigen-binding moieties of antibodies include variable fragment (Fv) and Fab fragments.

Antibodies generally comprise six complementarity-determining regions CDRs; three in the heavy chain variable (VH) region: HC-CDR1, HC-CDR2 and HC-CDR3, and three in the light chain variable (VL) region: LC-CDR1, LC-CDR2, and LC-CDR3. The six CDRs together define the paratope of the antibody, which is the part of the antibody which binds to the target antigen.

The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as those described in Kabat et al., Sequences of Proteins of Immunological Interest, 5t Ed. Public Health Service, National Institutes of Health, Bethesda, Md. (1991), Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibody clones described herein were defined according to the international IMGT (ImMunoGeneTics) information system (LeFranc et al., Nucleic Acids Res. (2015)43 (Database issue):D413-22), which uses the IMGT V-DOMAIN numbering rules as described in Lefranc et al., Dev. Comp. Immunol. (2003) 27:55-77.

In some embodiments, the antigen-binding molecule comprises the CDRs of an antigen-binding molecule which is capable of binding to a target antigen. In some embodiments, the antigen-binding molecule comprises the FRs of an antigen-binding molecule which is capable of binding to a target antigen. In some embodiments, the antigen-binding molecule comprises the CDRs and the FRs of an antigen-binding molecule which is capable of binding to a target antigen. That is, in some embodiments the antigen-binding molecule comprises the VH region and the VL region of an antigen-binding molecule which is capable of binding to a target antigen. In some embodiments, the antigen-binding molecule according to the present invention comprises an Fv region which binds to a target antigen. In some embodiments the Fv region comprises a polypeptide comprising a VH and a VL (e.g. a VH-VL fusion polypeptide). In some embodiments the VH and VL regions are provided as single polypeptide joined by a linker sequence (e.g. as described herein); i.e. as a single chain Fv (scFv).

In some embodiments, the antigen-binding molecule comprises a Fab fragment capable of binding to a target antigen. A Fab fragment comprises VH and VL regions, and further comprises immunoglobulin heavy chain constant region 1 (CH1) and immunoglobulin light chain constant region (CL). In some embodiments the antigen-binding molecule comprises a Fab region comprising a VH, a CH1, a VL and a CL (e.g. Cκ or Cλ). In some embodiments the Fab region comprises a polypeptide comprising a VH and a CH1 (e.g. a VH-CH1 fusion polypeptide), and a polypeptide comprising a VL and a CL (e.g. a VL-CL fusion polypeptide). In some embodiments the Fab region comprises a polypeptide comprising a VH and a CL (e.g. a VH-CL fusion polypeptide) and a polypeptide comprising a VL and a CH (e.g. a VL-CH1 fusion polypeptide); that is, in some embodiments the Fab region is a CrossFab region. In some embodiments the VH, CH1, VL and CL regions of the Fab or CrossFab are provided as single polypeptide joined by linker sequences; i.e. as a single chain Fab (scFab) or a single chain CrossFab (scCrossFab).

Different kinds of immunoglobulins and their structures are described e.g. in Schroeder and Cavacini J Allergy Clin Immunol. (2010) 125(202): S41-S52, which is hereby incorporated by reference in its entirety. Immunoglobulins of type G (i.e. IgG) are ˜150 kDa glycoproteins comprising two heavy chains and two light chains. From N- to C-terminus, the heavy chains comprise a VH followed by a heavy chain constant region comprising three constant domains (CH1, CH2, and CH3), and similarly the light chain comprise a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

Immunoglobulin heavy chain constant region sequences may be, or may be derived from, the heavy chain constant sequence of an IgG (e.g. IgG1, IgG2, IgG3, IgG4), IgA (e.g. IgA1, IgA2), IgD, IgE or IgM.

In some embodiments, heavy chain constant region sequences may be, or may be derived from, the heavy chain constant sequence of an IgG. In some embodiments, heavy chain constant region sequences may be, or may be derived from, the heavy chain constant sequence of a human IgG.

In some embodiments, heavy chain constant region sequences may be, or may be derived from, the heavy chain constant sequence of a human IgG1 allotype (e.g. G1m1, G1m2, G1m3 or G1m17).

In some embodiments the immunoglobulin heavy chain constant sequence is human immunoglobulin G 1 constant, G1m1 allotype (IGHG1; UniProt: P01857-1, v1; SEQ ID NO:1). Positions 1 to 98 of SEQ ID NO:1 form the CH1 region (SEQ ID NO:2). Positions 99 to 110 of SEQ ID NO:1 form a hinge region between CH1 and CH2 regions (SEQ ID NO:3). Positions 111 to 223 of SEQ ID NO:1 form the CH2 region (SEQ ID NO:4). Positions 224 to 330 of SEQ ID NO:1 form the CH3 region (SEQ ID NO:5).

In some embodiments the immunoglobulin heavy chain constant sequence is human immunoglobulin G 1 constant, G1m3 allotype (SEQ ID NO:28). Positions 1 to 98 of SEQ ID NO:28 form the CH1 region (SEQ ID NO:29). Positions 99 to 110 of SEQ ID NO:28 form a hinge region between CH1 and CH2 regions (SEQ ID NO:3). Positions 111 to 223 of SEQ ID NO:28 form the CH2 region (SEQ ID NO:4). Positions 224 to 330 of SEQ ID NO:28 form the CH3 region (SEQ ID NO:30).

In some embodiments, the antigen-binding molecule comprises one or more CH1 regions. In some embodiments a CH1 region comprises or consists of the sequence of SEQ ID NO:2, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO:2. In some embodiments a CH1 region comprises or consists of the sequence of SEQ ID NO:29, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO:29.

In some embodiments, the antigen-binding molecule comprises one or more CH1-CH2 hinge regions. In some embodiments a CH1-CH2 hinge region comprises or consists of the sequence of SEQ ID NO:3, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO:3.

In some embodiments the antigen-binding molecule of the present invention comprises one or more regions of an immunoglobulin light chain constant sequence. In some embodiments the immunoglobulin light chain constant sequence is human immunoglobulin kappa constant (IGKC; Cκ; UniProt: P01834-1, v2; SEQ ID NO:7). In some embodiments the immunoglobulin light chain constant sequence is a human immunoglobulin lambda constant (IGLC; Cλ), e.g. IGLC1, IGLC2, IGLC3, IGLC6 or IGLC7. In some embodiments, the antigen-binding molecule comprises one or more CL regions. In some embodiments a CL region comprises or consists of the sequence of SEQ ID NO:7, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO:7.

The antigen-binding molecules of the present invention may be provided with any suitable format, provided they comprise at least one antigen-binding moiety and at least one Fc region.

In some embodiments, the antigen-binding molecule comprises an antigen-binding moiety which comprises, or consists of, one of: an Fv, scFv, Fab, scFab, CrossFab, scCrossFab, VhH, diabody or triabody specific for a target antigen.

In some embodiments, the antigen-binding molecule comprises more than one (e.g. 2, 3, 4, 5, 6, 7, 8, etc.) antigen-binding moiety. In embodiments wherein the antigen-binding molecule comprises more than one antigen-binding moiety, the antigen-binding moieties may be identical or non-identical. In some embodiments the antigen-binding moieties may each independently be an antigen-binding moiety according to any embodiment of an antigen-binding moiety as described herein.

In some embodiments the antigen-binding molecule is monospecific (i.e. comprises one type of antigen-binding moiety). By way of illustration the antigen-binding molecules characterised in the experimental examples of the present disclosure are monospecific for HER3.

In some embodiments the antigen-binding molecule is multispecific (e.g. bispecific, trispecific, etc.); that is, in some embodiments the antigen-binding molecule comprises more than one (e.g. 2, 3, etc.) different types of antigen-binding moiety. Multispecific antigen-binding molecules may bind to non-identical epitopes. Multispecific antigen-binding molecules may bind to non-identical target antigens.

The antigen-binding molecule according to the present invention displays at least monovalent binding to a target antigen. Binding valency refers to the number of binding sites in an antigen-binding molecule for a given target. In some embodiments, the antigen-binding molecule is multivalent (e.g. bivalent, trivalent etc.) for a given target. By way of illustration the antigen-binding molecules characterised in the experimental examples of the present disclosure are comprise two identical binding sites for HER3, and are thus monospecific, bivalent antigen-binding molecules.

In addition to the moiety capable of binding to a target antigen, the antigen-binding molecules of the present invention comprise an Fc region.

The target antigen for which the antigen-binding moiety (and thus the antigen-binding molecule) is specific may be any target antigen. In some embodiments, the target antigen is an antigen whose expression/activity, or whose upregulated expression/activity, is positively associated with a disease or disorder (e.g. a cancer, an infectious disease or an autoimmune disease). In some embodiments the target antigen is expressed at the cell surface of a cell expressing the target antigen.

In some embodiments, the target antigen is expressed by an infectious agent, cell, or a cell of a tissue, which it is desirable to destroy or remove. In some embodiments, the target antigen is expressed by a pathogen, cell, or a cell of a tissue to which it is desirable to direct an immune response, e.g. a humoral or cell mediated immune response. In some embodiments the target antigen is associated with a cancer, an infectious disease, or an autoimmune disease.

In some embodiments, the target antigen is expressed by a cancer cell, an infectious agent, a cell infected with an infectious agent or an autoimmune effector cell (i.e. an effector of an autoimmune pathology).

In some embodiments the target antigen is a cancer cell antigen (i.e. an antigen which is expressed or over-expressed by a cancer cell). A cancer cell antigen's expression may be associated with a cancer. A cancer cell antigen may be abnormally expressed by a cancer cell (e.g. the cancer cell antigen may be expressed with abnormal localisation), or may be expressed with an abnormal structure by a cancer cell. A cancer cell antigen may be capable of eliciting an immune response. In some embodiments, the antigen is expressed at the cell surface of the cancer cell (i.e. the cancer cell antigen is a cancer cell surface antigen). In some embodiments, the part of the antigen which is bound by the antigen-binding molecule described herein is displayed on the external surface of the cancer cell (i.e. is extracellular). The cancer cell antigen may be a cancer-associated antigen. In some embodiments the cancer cell antigen is an antigen whose expression is associated with the development, progression or severity of symptoms of a cancer. The cancer-associated antigen may be associated with the cause or pathology of the cancer, or may be expressed abnormally as a consequence of the cancer. In some embodiments, the cancer cell antigen is an antigen whose expression is upregulated (e.g. at the RNA and/or protein level) by cells of a cancer, e.g. as compared to the level of expression of by comparable non-cancerous cells (e.g. non-cancerous cells derived from the same tissue/cell type). In some embodiments, the cancer-associated antigen may be preferentially expressed by cancerous cells, and not expressed by comparable non-cancerous cells (e.g. non-cancerous cells derived from the same tissue/cell type). In some embodiments, the cancer-associated antigen may be the product of a mutated oncogene or mutated tumor suppressor gene. In some embodiments, the cancer-associated antigen may be the product of an overexpressed cellular protein, a cancer antigen produced by an oncogenic virus, an oncofetal antigen, or a cell surface glycolipid or glycoprotein.

In some embodiments the target antigen is an antigen of an infectious agent. The antigen may be an antigen which is expressed by the infectious agent. The antigen may be an antigen which is presented at the cell surface of a cell infected with the infectious agent. The antigen may be an antigen which is presented at the cell surface of a cell which has internalized (e.g. phagocytosed) the infectious agent. The antigen may be an antigen whose expression is associated with infection by an infectious agent, e.g. an antigen which is abnormally expressed by a cell infected with an infectious agent. In some embodiments, the target antigen is an antigen whose expression is upregulated (e.g. at the RNA and/or protein level) by cells infected with an infectious agent, e.g. as compared to the level of expression of by comparable non-infected cells (e.g. non-infected cells derived from the same tissue/cell type). In some embodiments, the target antigen may be preferentially expressed by cells infected with an infectious agent, and not expressed by comparable non-infected cells (e.g. non-infected cells derived from the same tissue/cell type).

In some embodiments the target antigen is of an autoimmune effector cell (i.e. an antigen which is expressed or over-expressed by an autoimmune effector cell). The antigen's expression may be associated with an autoimmune pathology. In some embodiments, the antigen is expressed at the cell surface of an autoimmune effector cell. In some embodiments, the part of the antigen which is bound by the antigen-binding molecule described herein is displayed on the external surface of the an autoimmune effector cell (i.e. is extracellular). In some embodiments the antigen is an antigen whose expression is associated with the development, progression or severity of symptoms of an autoimmune disease/condition. The antigen may be associated with the cause or pathology of the autoimmune disease/condition. In some embodiments, the antigen's expression is upregulated (e.g. at the RNA and/or protein level) by autoimmune effector cells, e.g. as compared to cells derived from the same tissue/cell type which are not autoimmune effector cells. In some embodiments, the antigen may be preferentially expressed by autoimmune effector cells, and not expressed by comparable cells which are not autoimmune effector cells.

In some embodiments the target antigen is HERS. In some embodiments the target antigen is VISTA. In some embodiments the target antigen is CD47. In some embodiments the target antigen is CD33. In some embodiments the target antigen is BCMA. In some embodiments the target antigen is TACI.

Fc Regions

The present invention provides antigen-binding molecules comprising an Fc region. Also provided are Fc regions. Fc regions may be provided in isolated or substantially purified form.

Fc regions provide for interaction with Fc receptors and other molecules of the immune system to bring about functional effects. IgG Fc-mediated effector functions are reviewed e.g. in Jefferis et al., Immunol Rev 1998 163:59-76 (hereby incorporated by reference in its entirety), and are brought about through Fc-mediated recruitment and activation of immune cells (e.g. macrophages, dendritic cells, neutrophils, basophils, eosinophils, platelets, mast cells, NK cells and T cells) through interaction between the Fc region and Fc receptors expressed by the immune cells, recruitment of complement pathway components through binding of the Fc region to complement protein C1q, and consequent activation of the complement cascade.

In IgG IgA and IgD isotypes, Fc regions are composed of CH2 and CH3 regions from one polypeptide, and CH2 and CH3 regions from another polypeptide. The CH2 and CH3 regions from the two polypeptides together form the Fc region. In IgM and IgE isotypes the Fc regions contain three constant domains (CH2, CH3 and CH4), and CH2 to CH4 from the two polypeptides together form the Fc region.

In preferred embodiments in accordance with the various aspects of the present disclosure an Fc region comprises, two polypeptides, each polypeptide comprising a CH2 region and a CH3 region.

Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

Modifications to antibody Fc regions that influence Fc-mediated functions are known in the art, such as those described e.g. in Wang et al., Protein Cell (2018) 9(1):63-73, which is hereby incorporated by reference in its entirety. Exemplary Fc region modifications known to influence antibody effector function are summarised in Table 1 of Wang et al., Protein Cell (2018) 9(1):63-73.

The combination of substitutions F243L/R292P/Y300L/V305I/P396L is described in Stavenhagen et al. Cancer Res. (2007) to increase binding to FcγRIIIa, and thereby enhance ADCC. The combination of substitutions S239D/1332E or S239D/1332E/A330L is described in Lazar et al., Proc Natl Acad Sci USA. (2006)103:4005-4010 to increase binding to FcγRIIIa, and thereby increase ADCC. The combination of substitutions S239D/1332E/A330L is also described to decrease binding to FcγRIIb, and thereby increase ADCC. The combination of substitutions S298A/E333A/K334A is described in Shields et al., J Biol Chem. (2001)276:6591-6604 to increase binding to FcγRIIIa, and thereby increase ADCC. The combination of substitutions L234Y/L235Q/G236W/S239M/H268D/D270E/S298A in one heavy chain, and the combination of substitutions D270E/K326D/A330M/K334E in the other heavy chain, is described in Mimoto et al., MAbs. (2013): 5:229-236 to increase binding to FcγRIIIa, and thereby increase ADCC. The combination of substitutions G236A/S239D/I332E is described in Richards et al., Mol Cancer Ther. (2008) 7:2517-2527 to increase binding to FcγRIIa and to increase binding to FcγRIIIa, and thereby increase ADCP.

The combination of substitutions K326W/E333S is described in Idusogie et al. J Immunol. (2001) 166(4):2571-5 to increase binding to C1q, and thereby increase CDC. The combination of substitutions S267E/H268F/S324T is described in Moore et al. MAbs. (2010) 2(2):181-9 to increase binding to C1q, and thereby increase CDC. The combination of substitutions described in Natsume et al., Cancer Res. (2008) 68(10):3863-72 is reported to increase binding to C1q, and thereby increase CDC. The combination of substitutions E345R/E430G/S440Y is described in Diebolder et al. Science (2014) 343(6176):1260-3 to increase hexamerisation, and thereby increase CDC.

The combination of substitutions E345K/E430G is described in de Jong et al. PLoS Biol. (2016) 14(1):e1002344 to increase hexamer formation and complement activation, and thereby increase CDC.

The combination of substitutions M252Y/S254T/T256E is described in Dall'Acqua et al. J Immunol. (2002) 169:5171-5180 to increase binding to FcRn at pH 6.0, and thereby increase antigen-binding molecule half-life. The combination of substitutions M428L/N434S is described in Zalevsky et al. Nat Biotechnol. (2010) 28:157-159 to increase binding to FcRn at pH 6.0, and thereby increase antigen-binding molecule half-life.

The combination of substitutions L242C, K334C is described in Gong et al. J Biol Chem. (2009) 284(21):14203-10 and McConnell et al. Protein Eng Des Sel. (2013) 26(2):151-64 to increase thermal stability of antibody constant domains.

Where a heavy chain constant region/Fc region/CH2-CH3 region/CH2 region/CH3 region is described herein as comprising position(s)/substitution(s) “corresponding to” reference position(s)/substitution(s), equivalent position(s)/substitution(s) in homologous heavy chain constant regions/Fc regions/CH2-CH3 regions/CH2 regions/CH3 regions are contemplated.

Where an Fc region is described as comprising specific position(s)/substitution(s), the position(s)/substitution(s) may be present in one or both of the polypeptide chains which together form the Fc region.

Unless otherwise specified, positions herein refer to positions of human immunoglobulin constant region amino acid sequences numbered according to the EU numbering system as described in Kabat et al., Sequences of Proteins of Immunological Interest, 5t Ed. Public Health Service, National Institutes of Health, Bethesda, Md., 1991.

Homologous heavy chain constant regions to human IgG1(G1m1) heavy chain constant region are heavy chain constant regions comprising an amino acid sequence having at least 60%, preferably one of 70%, 75%, 80%, 85%, 90%, 91%, 92%, 93%, 94%, 95%, 96%, 97%, 98%, 99% or 100% amino acid sequence identity to the heavy chain constant region of Human IgG1(G1m1) (i.e. the amino acid sequence shown in SEQ ID NO:1). Homologous Fc regions to human IgG1(G1m1) Fc region are Fc regions comprised of polypeptides comprising an amino acid sequence having at least 60%, preferably one of 70%, 75%, 80%, 85%, 90%, 91%, 92%, 93%, 94%, 95%, 96%, 97%, 98%, 99% or 100% amino acid sequence identity to CH2-CH3 region of Human IgG1(G1m1) (i.e. the amino acid sequence shown in SEQ ID NO:6). Homologous CH2 regions to human IgG1(G1m1) CH2 region are CH2 regions comprising an amino acid sequence having at least 60%, preferably one of 70%, 75%, 80%, 85%, 90%, 91%, 92%, 93%, 94%, 95%, 96%, 97%, 98%, 99% or 100% amino acid sequence identity to CH2 region of Human IgG1(G1m1) (i.e. the amino acid sequence shown in SEQ ID NO:4). Homologous CH3 regions to human IgG1(G1m1) CH3 region are CH3 regions comprising an amino acid sequence having at least 60%, preferably one of 70%, 75%, 80%, 85%, 90%, 91%, 92%, 93%, 94%, 95%, 96%, 97%, 98%, 99% or 100% amino acid sequence identity to CH3 region of Human IgG1(G1 m1) (i.e. the amino acid sequence shown in SEQ ID NO:5).

Homologous heavy chain constant regions to human IgG1(G1m3) heavy chain constant region are heavy chain constant regions comprising an amino acid sequence having at least 60%, preferably one of 70%, 75%, 80%, 85%, 90%, 91%, 92%, 93%, 94%, 95%, 96%, 97%, 98%, 99% or 100% amino acid sequence identity to the heavy chain constant region of Human IgG1(G1m3) (i.e. the amino acid sequence shown in SEQ ID NO:28). Homologous Fc regions to human IgG1(G1m3) Fc region are Fc regions comprised of polypeptides comprising an amino acid sequence having at least 60%, preferably one of 70%, 75%, 80%, 85%, 90%, 91%, 92%, 93%, 94%, 95%, 96%, 97%, 98%, 99% or 100% amino acid sequence identity to CH2-CH3 region of Human IgG1(G1m3) (i.e. the amino acid sequence shown in SEQ ID NO:31). Homologous CH2 regions to human IgG1(G1m3) CH2 region are CH2 regions comprising an amino acid sequence having at least 60%, preferably one of 70%, 75%, 80%, 85%, 90%, 91%, 92%, 93%, 94%, 95%, 96%, 97%, 98%, 99% or 100% amino acid sequence identity to CH2 region of Human IgG1(G1m3) (i.e. the amino acid sequence shown in SEQ ID NO:4). Homologous CH3 regions to human IgG1(G1m3) CH3 region are CH3 regions comprising an amino acid sequence having at least 60%, preferably one of 70%, 75%, 80%, 85%, 90%, 91%, 92%, 93%, 94%, 95%, 96%, 97%, 98%, 99% or 100% amino acid sequence identity to CH3 region of Human IgG1(G1 m3) (i.e. the amino acid sequence shown in SEQ ID NO:30).

Corresponding positions to those identified in human IgG1 can be identified by sequence alignment which can be performed e.g. using sequence alignment software such as ClustalOmega (Söding, J. 2005, Bioinformatics 21, 951-960).

By way of illustration, the substitutions L242C and K334C in human IgG1 correspond to I>C substitution at position 125, and R>C substitution at position 217 of the mouse Ig gamma-2A chain C region, A allele, numbered according to SEQ ID NO:25.

In some embodiments the Fc region comprises modification to increase an Fc-mediated function. In some embodiments the Fc region comprises modification to increase ADCC. In some embodiments the Fc region comprises modification to increase ADCP. In some embodiments the Fc region comprises modification to increase or decrease CDC. An antigen-binding molecule comprising an Fc region comprising modification to increase an Fc-mediated function (e.g. ADCC, ADCP, CDC) induces an increased level of the relevant effector function as compared to an antigen-binding molecule comprising the corresponding unmodified Fc region. An antigen-binding molecule comprising an Fc region comprising modification to decrease an Fc-mediated function (e.g. CDC) induces a decreased level of the relevant effector function as compared to an antigen-binding molecule comprising the corresponding unmodified Fc region.

In some embodiments the Fc region comprises modification to increase binding to an Fc receptor. In some embodiments the Fc region comprises modification to increase binding to an Fcγ receptor. In some embodiments the Fc region comprises modification to increase binding to one or more of FcγRI, FcγRIIa, FcγRIIb, FcγRIIc, FcγRIIIa and FcγRIIIb. In some embodiments the Fc region comprises modification to increase binding to FcγRIIIa. In some embodiments the Fc region comprises modification to increase binding to FcγRIIa. In some embodiments the Fc region comprises modification to increase binding to FcγRIIb. In some embodiments the Fc region comprises modification to decrease binding to FcγRIIb. In some embodiments the Fc region comprises modification to increase binding to FcRn. In some embodiments the Fc region comprises modification to increase binding to a complement protein. In some embodiments the Fc region comprises modification to increase or decrease binding to C1q. In some embodiments the Fc region comprises modification to promote hexamerisation of the antigen-binding molecule. In some embodiments the Fc region comprises modification to increase antigen-binding molecule half-life. In some embodiments the Fc region comprises modification to increase co-engagement.

In some embodiments the Fc region comprises (e.g. comprises one more polypeptides comprising a heavy chain constant region, or a CH2-CH3 region, comprising) one or more (e.g. 1, 2, 3, 4, 5, 6, 7 or 8) of the following: C at the position corresponding to position 242; C at the position corresponding to position 334; A at the position corresponding to position 236; D at the position corresponding to position 239; E at the position corresponding to position 332; L at the position corresponding to position 330; K at the position corresponding to position 345; and G at the position corresponding to position 430.

In some embodiments the Fc region comprises (e.g. comprises one more polypeptides comprising a heavy chain constant region, or a CH2-CH3 region, comprising) one or more (e.g. 1, 2, 3, 4, 5, 6, 7 or 8) of the following substitutions (or corresponding substitutions): L242C, K334C, G236A, S239D, I332E, A330L, E345K, and E430G.

In some embodiments the Fc region comprises (e.g. comprises one more polypeptides comprising a heavy chain constant region, a CH2-CH3 region, or a CH2 region, comprising) a C at the position corresponding to position 242. In some embodiments the Fc region comprises (e.g. comprises one more polypeptides comprising a heavy chain constant region, a CH2-CH3 region, or a CH2 region, comprising) a C at the position corresponding to position 334. In some embodiments the Fc region comprises (e.g. comprises one more polypeptides comprising a heavy chain constant region, a CH2-CH3 region, or a CH2 region, comprising) a C at the position corresponding to position 242 and a C at the position corresponding to position 334.

In some embodiments the Fc region comprises (e.g. comprises one more polypeptides comprising a heavy chain constant region, a CH2-CH3 region, or a CH2 region, comprising) an A at the position corresponding to position 236. In some embodiments the Fc region comprises (e.g. comprises one more polypeptides comprising a heavy chain constant region, a CH2-CH3 region, or a CH2 region, comprising) a D at the position corresponding to position 239. In some embodiments the Fc region comprises (e.g. comprises one more polypeptides comprising a heavy chain constant region, a CH2-CH3 region, or a CH2 region, comprising) an A at the position corresponding to position 236, and a D at the position corresponding to position 239.

In some embodiments the Fc region comprises (e.g. comprises one more polypeptides comprising a heavy chain constant region, a CH2-CH3 region, or a CH2 region, comprising) an E at the position corresponding to position 332. In some embodiments the Fc region comprises (e.g. comprises one more polypeptides comprising a heavy chain constant region, a CH2-CH3 region, or a CH2 region, comprising) an A at the position corresponding to position 236, a D at the position corresponding to position 239, and an E at the position corresponding to position 332.

In some embodiments the Fc region comprises (e.g. comprises one more polypeptides comprising a heavy chain constant region, a CH2-CH3 region, or a CH2 region, comprising) an L at the position corresponding to position 330. In some embodiments the Fc region comprises (e.g. comprises one more polypeptides comprising a heavy chain constant region, a CH2-CH3 region, or a CH2 region, comprising) an A at the position corresponding to position 236, a D at the position corresponding to position 239, an E at the position corresponding to position 332, and an L at the position corresponding to position 330.

In some embodiments the Fc region comprises (e.g. comprises one more polypeptides comprising a heavy chain constant region, a CH2-CH3 region, or a CH3 region, comprising) a K at the position corresponding to position 345. In some embodiments the Fc region comprises (e.g. comprises one more polypeptides comprising a heavy chain constant region, a CH2-CH3 region, or a CH3 region, comprising) a G at the position corresponding to position 430. In some embodiments the Fc region comprises (e.g. comprises one more polypeptides comprising a heavy chain constant region, a CH2-CH3 region, or a CH3 region, comprising) a K at the position corresponding to position 345, and a G at the position corresponding to position 430.

In some embodiments the Fc region comprises (e.g. comprises one more polypeptides comprising a heavy chain constant region, a CH2-CH3 region, or a CH2 region, comprising) a C at the position corresponding to position 242, a C at the position corresponding to position 334, an A at the position corresponding to position 236, and a D at the position corresponding to position 239.

In some embodiments the Fc region comprises (e.g. comprises one more polypeptides comprising a heavy chain constant region, a CH2-CH3 region, or a CH2 region, comprising) a C at the position corresponding to position 242, a C at the position corresponding to position 334, an A at the position corresponding to position 236, a D at the position corresponding to position 239, and an E at the position corresponding to position 332.

In some embodiments the Fc region comprises (e.g. comprises one more polypeptides comprising a heavy chain constant region, a CH2-CH3 region, or a CH2 region, comprising) a C at the position corresponding to position 242, a C at the position corresponding to position 334, an A at the position corresponding to position 236, a D at the position corresponding to position 239, an E at the position corresponding to position 332, and an L at the position corresponding to position 330.

In some embodiments the Fc region comprises (e.g. comprises one more polypeptides comprising a heavy chain constant region, or a CH2-CH3 region, comprising) a C at the position corresponding to position 242, a C at the position corresponding to position 334, a K at the position corresponding to position 345, and a G at the position corresponding to position 430.

In some embodiments the Fc region comprises (e.g. comprises one more polypeptides comprising a heavy chain constant region, a CH2-CH3 region, or a CH2 region, comprising) the substitution L242C (or an equivalent substitution). In some embodiments the Fc region comprises (e.g. comprises one more polypeptides comprising a heavy chain constant region, a CH2-CH3 region, or a CH2 region, comprising) the substitution K334C (or an equivalent substitution). In some embodiments the Fc region comprises (e.g. comprises one more polypeptides comprising a heavy chain constant region, a CH2-CH3 region, or a CH2 region, comprising) the substitution L242C (or an equivalent substitution) and the substitution K334C (or an equivalent substitution).

In some embodiments the Fc region comprises (e.g. comprises one more polypeptides comprising a heavy chain constant region, a CH2-CH3 region, or a CH2 region, comprising) the substitution G236A (or an equivalent substitution). In some embodiments the Fc region comprises (e.g. comprises one more polypeptides comprising a heavy chain constant region, a CH2-CH3 region, or a CH2 region, comprising) the substitution S239D (or an equivalent substitution). In some embodiments the Fc region comprises (e.g. comprises one more polypeptides comprising a heavy chain constant region, a CH2-CH3 region, or a CH2 region, comprising) the substitution G236A (or an equivalent substitution), and the substitution S239D (or an equivalent substitution).

In some embodiments the Fc region comprises (e.g. comprises one more polypeptides comprising a heavy chain constant region, a CH2-CH3 region, or a CH2 region, comprising) the substitution I332E (or an equivalent substitution). In some embodiments the Fc region comprises (e.g. comprises one more polypeptides comprising a heavy chain constant region, a CH2-CH3 region, or a CH2 region, comprising) the substitution G236A (or an equivalent substitution), the substitution S239D (or an equivalent substitution), and the substitution I332E (or an equivalent substitution).

In some embodiments the Fc region comprises (e.g. comprises one more polypeptides comprising a heavy chain constant region, a CH2-CH3 region, or a CH2 region, comprising) the substitution A330L (or an equivalent substitution). In some embodiments the Fc region comprises (e.g. comprises one more polypeptides comprising a heavy chain constant region, a CH2-CH3 region, or a CH2 region, comprising) the substitution G236A (or an equivalent substitution), the substitution S239D (or an equivalent substitution), the substitution I332E (or an equivalent substitution), and the substitution A330L (or an equivalent substitution).

In some embodiments the Fc region comprises (e.g. comprises one more polypeptides comprising a heavy chain constant region, a CH2-CH3 region, or a CH3 region, comprising) the substitution E345K (or an equivalent substitution). In some embodiments the Fc region comprises (e.g. comprises one more polypeptides comprising a heavy chain constant region, a CH2-CH3 region, or a CH3 region, comprising) the substitution E430G (or an equivalent substitution). In some embodiments the Fc region comprises (e.g. comprises one more polypeptides comprising a heavy chain constant region, a CH2-CH3 region, or a CH3 region, comprising) the substitution E345K (or an equivalent substitution), and the substitution E430G (or an equivalent substitution).

In some embodiments the Fc region comprises (e.g. comprises one more polypeptides comprising a heavy chain constant region, a CH2-CH3 region, or a CH2 region, comprising) the substitution L242C (or an equivalent substitution), the substitution K334C (or an equivalent substitution), the substitution G236A (or an equivalent substitution), and the substitution S239D (or an equivalent substitution).

In some embodiments the Fc region comprises (e.g. comprises one more polypeptides comprising a heavy chain constant region, a CH2-CH3 region, or a CH2 region, comprising) the substitution L242C (or an equivalent substitution), the substitution K334C (or an equivalent substitution), the substitution G236A (or an equivalent substitution), the substitution S239D (or an equivalent substitution), and the substitution I332E (or an equivalent substitution).

In some embodiments the Fc region comprises (e.g. comprises one more polypeptides comprising a heavy chain constant region, a CH2-CH3 region, or a CH2 region, comprising) the substitution L242C (or an equivalent substitution), the substitution K334C (or an equivalent substitution), the substitution G236A (or an equivalent substitution), the substitution S239D (or an equivalent substitution), the substitution I332E (or an equivalent substitution), and the substitution A330L (or an equivalent substitution).

In some embodiments the Fc region comprises (e.g. comprises one more polypeptides comprising a heavy chain constant region, or a CH2-CH3 region, comprising) the substitution L242C (or an equivalent substitution), the substitution K334C (or an equivalent substitution), the substitution E345K (or an equivalent substitution), and the substitution E430G (or an equivalent substitution).

In some embodiments the Fc region comprises one or more polypeptides comprising an amino acid sequence having at least 60%, preferably one of 70%, 75%, 80%, 85%, 90%, 91%, 92%, 93%, 94%, 95%, 96%, 97%, 98%, 99% or 100% sequence identity to SEQ ID NO:8, 9, 10, 11, 12, 13, 14 or 15.

In some embodiments the Fc region comprises one or more polypeptides comprising an amino acid sequence having at least 60%, preferably one of 70%, 75%, 80%, 85%, 90%, 91%, 92%, 93%, 94%, 95%, 96%, 97%, 98%, 99% or 100% sequence identity to SEQ ID NO:16, 17, 18, 19, 20, 21, 22, 23, 24, 33, 34, 35, 36, 37, 38, 39, 40 or 41.

In some embodiments the Fc region comprises (e.g. comprises one more polypeptides comprising a heavy chain constant region, or a CH2-CH3 region, comprising) one or more (e.g. 1, 2, 3, 4, 5, 6, 7, 8, 9, 10, 11 or 12) of the following: L at the position corresponding to position 243, P at the position corresponding to position 292, L at the position corresponding to position 300, I at the position corresponding to position 305 and L at the position corresponding to position 396; D at the position corresponding to position 239 and E at the position corresponding to position 332; D at the position corresponding to position 239, E at the position corresponding to position 332 and L at the position corresponding to position 330; A at the position corresponding to position 298, A at the position corresponding to position 333 and A at the position corresponding to position 334; Y at the position corresponding to position 234, Q at the position corresponding to position 235, W at the position corresponding to position 236, M at the position corresponding to position 239, D at the position corresponding to position 268, E at the position corresponding to position 270 and A at the position corresponding to position 298; E at the position corresponding to position 270, D at the position corresponding to position 326, M at the position corresponding to position 330 and E at the position corresponding to position 334; A at the position corresponding to position 236, D at the position corresponding to position 239 and E at the position corresponding to position 332; W at the position corresponding to position 326 and S at the position corresponding to position 333; E at the position corresponding to position 267, F at the position corresponding to position 268 and T at the position corresponding to position 324; R at the position corresponding to position 345, G at the position corresponding to position 430 and Y at the position corresponding to position 440; Y at the position corresponding to position 252, T at the position corresponding to position 254 and E at the position corresponding to position 256; and L at the position corresponding to position 428 and S at the position corresponding to position 434.

In some embodiments the Fc region comprises (e.g. comprises one more polypeptides comprising a heavy chain constant region, or a CH2-CH3 region, comprising) one or more (e.g. 1, 2, 3, 4, 5, 6, 7, 8, 9, 10, 11 or 12) of the following combinations of substitutions (or corresponding substitutions): F243L/R292P/Y300/V305I/P396L; S239D/I332E; S239D/I332E/A330L; S298A/E333A/K334A; L234Y/L235Q/G236W/S239M/H268D/D270E/S298A; D270E/K326D/A330M/K334E; G236A/S239D/I332E; K326W/E333S; S267E/H268F/S324T; E345R/E430G/S440Y; M252Y/S254T/T256E; and M428L/N434S.

In some embodiments the Fc region or polypeptide(s) comprise modification to oligosaccharide attached to the Fc region (relative to the oligosaccharide attached to the Fc region of wildtype IgG1). Glycoengineering of Fc regions is described e.g. in Kellner et al., Transfus Med Hemother (2017) 44:327-336, which is hereby incorporated by reference in its entirety. The presence of the oligosaccharide attached to of N297 human IgG1 Fc is important for binding to Fcγ receptors and the C1q, and reduced fucose or sialic acid content in the N-glycan has been shown to improve ADCC activity.

Various different approaches have been used to prepare antibodies with glycoengineered Fc regions, including production from cells deficient for the activity of one or more enzymes involved in glycan processing/modification. For example, Fc with reduced fucose in the N-glycan can be prepared by expression from cells modified for reduced protein expression of a fucosyltransferase (e.g. by gene knockout or antisense interference). Alternatively, antibodies can be treated with factors for removing fucose residues. Other approaches to modification of antibody Fc glycans include expression from cells having upregulated expression of N-acetylglucosaminyltransferase, for the production of N-glycans having a bisecting GlcNAc, which improves Fc receptor binding.

In some embodiments the Fc region or polypeptide(s) comprise N-glycan lacking fucose. In some embodiments the Fc region or polypeptide(s) comprise N-glycan lacking sialic acid. In some embodiments the Fc region or polypeptide(s) lack N-glycan comprising fucose. In some embodiments the Fc region or polypeptide(s) lack N-glycan comprising sialic acid. In some embodiments the Fc region or polypeptide(s) comprise N-glycan lacking fucose. In some embodiments the Fc region or polypeptide(s) comprise N-glycan comprising a bisecting GlcNAc.

In some embodiments, the Fc regions comprise modification in one or more of the CH2 and CH3 regions promoting association of the constituent polypeptides of the Fc region. Recombinant co-expression of constituent polypeptides of an antigen-binding molecule and subsequent association leads to several possible combinations. To improve the yield of the desired combinations of polypeptides in antigen-binding molecules in recombinant production, it is advantageous to introduce in the Fc regions modification(s) promoting association of the desired combination of heavy chain polypeptides. Modifications may promote e.g. hydrophobic and/or electrostatic interaction between CH2 and/or CH3 regions of different polypeptide chains. Suitable modifications are described e.g. in Ha et al., Front. Immnol (2016) 7:394, which is hereby incorporated by reference in its entirety.

In some embodiments the antigen antigen-binding molecule of the present invention comprises an Fc region comprising paired substitutions in the CH3 regions of the Fc region according to one of the following formats, as shown in Table 1 of Ha et al., Front. Immnol (2016) 7:394: KiH, KiHs-s, HA-TF, ZW1, 7.8.60, DD-KK, EW-RVT, EW-RVTs-s, SEED or A107. In some embodiments the antigen antigen-binding molecule of the present invention comprises an Fc region comprising paired substitutions in the CH3 regions of the Fc region according to one of the following formats, as shown in Table 1 of Brinkmann and Kontermann, MABS (2017) 9(2):182-212 (hereby incorporated by reference in its entirety).

In some embodiments, the Fc region comprises the “knob-into-hole” or “KiH” modification, e.g. as described e.g. in U.S. Pat. No. 7,695,936 and Carter, J Immunol Meth 248, 7-15 (2001). In such embodiments, one of the CH3 regions of the Fc region comprises a “knob” modification, and the other CH3 region comprises a “hole” modification. The “knob” and “hole” modifications are positioned within the respective CH3 regions so that the “knob” can be positioned in the “hole” in order to promote heterodimerisation (and inhibit homodimerisation) of the polypeptides and/or stabilise heterodimers. Knobs are constructed by substituting amino acids having small chains with those having larger side chains (e.g. tyrosine or tryptophan). Holes are created by substituting amino acids having large side chains with those having smaller side chains (e.g. alanine or threonine).

In some embodiments, one of the CH3 regions of the Fc region of the antigen-binding molecule of the present invention comprises the substitution (numbering of positions/substitutions in the Fc, CH2 and CH3 regions herein is according to the EU numbering system as described in Kabat et al., Sequences of Proteins of Immunological Interest, 5t Ed. Public Health Service, National Institutes of Health, Bethesda, Md., 1991) T366W, and the other CH3 region of the Fc region comprises the substitution Y407V. In some embodiments, one of the CH3 regions of the Fc region of the antigen-binding molecule comprises the substitution T366W, and the other CH3 region of the Fc region comprises the substitutions T366S and L368A. In some embodiments, one of the CH3 regions of the Fc region of the antigen-binding molecule comprises the substitution T366W, and the other CH3 region of the Fc region comprises the substitutions Y407V, T366S and L368A.

In some embodiments, the Fc region comprises the “DD-KK” modification as described e.g. in WO 2014/131694 A1. In some embodiments, one of the CH3 regions comprises the substitutions K392D and K409D, and the other CH3 region of the Fc region comprises the substitutions E356K and D399K. The modifications promote electrostatic interaction between the CH3 regions.

In some embodiments, the antigen-binding molecule of the present invention comprises an Fc region modified as described in Labrijn et al., Proc Natl Acad Sci U S A. (2013) 110(13):5145-50, referred to as ‘Duobody’ format. In some embodiments one of the CH3 regions comprises the substitution K409R, and the other CH3 region of the Fc region comprises the substitution K405L.

In some embodiments, the antigen-binding molecule of the present invention comprises an Fc region comprising the “EEE-RRR” modification as described in Strop et al., J Mol Biol. (2012) 420(3):204-19. In some embodiments one of the CH3 regions comprises the substitutions D221E, P228E and L368E, and the other CH3 region of the Fc region comprises the substitutions D221R, P228R and K409R.

In some embodiments, the antigen-binding molecule comprises an Fc region comprising the “EW-RVT” modification described in Choi et al., Mol Cancer Ther (2013) 12(12):2748-59. In some embodiments one of the CH3 regions comprises the substitutions K360E and K409W, and the other CH3 region of the Fc region comprises the substitutions Q347R, D399V and F405T.

In some embodiments, one of the CH3 regions comprises the substitution S354C, and the other CH3 region of the Fc region comprises the substitution Y349C. Introduction of these cysteine residues results in formation of a disulphide bridge between the two CH3 regions of the Fc region, further stabilizing the heterodimer (Carter (2001), J Immunol Methods 248, 7-15).

In some embodiments, the Fc region comprises the “KiHS-S” modification. In some embodiments one of the CH3 regions comprises the substitutions T366W and S354C, and the other CH3 region of the Fc region comprises the substitutions T366S, L368A, Y407V and Y349C.

In some embodiments, the antigen-binding molecule of the present invention comprises an Fc region comprising the “SEED” modification as described in Davis et al., Protein Eng Des Sel (2010) 23(4):195-202, in which n-strand segments of human IgG1 CH3 and IgA CH3 are exchanged.

In some embodiments, one of the CH3 regions comprises the substitutions S364H and F405A, and the other CH3 region of the Fc region comprises the substitutions Y349T and I394F (see e.g. Moore et al., MAbs (2011) 3(6):546-57).

In some embodiments, one of the CH3 regions comprises the substitutions T350V, L351Y, F405A and Y407V, and the other CH3 region of the Fc region comprises the substitutions T350V, T366L, K392L and I394W (see e.g. Von Kreudenstein et al., MAbs (2013) 5(5):646-54).

In some embodiments, one of the CH3 regions comprises the substitutions K360D, D399M and Y407A, and the other CH3 region of the Fc region comprises the substitutions E345R, Q347R, T366V and K409V (see e.g. Leaver-Fay et al., Structure (2016) 24(4):641-51).

In some embodiments, one of the CH3 regions comprises the substitutions K370E and K409W, and the other CH3 region of the Fc region comprises the substitutions E357N, D399V and F405T (see e.g. Choi et al., PLoS One (2015) 10(12):e0145349).

Fc Receptors

Fc receptors are polypeptides which bind to the Fc region of immunoglobulins. Fc receptor structure and function is reviewed e.g. in Masuda et al., Inflamm Allergy Drug Targets (2009) 8(1): 80-86, and Bruhns, Blood (2012) 119:5640-5649, both of which are hereby incorporated by reference in their entirety.

Fc receptors are expressed at surface of hematopoietic cells including macrophages, neutrophils, dendritic cells, eosinophils, basophils, mast cells, and NK cells. They include the IgG-binding Fcγ receptors, the high-affinity receptor for IgE (FcεRI), the IgA receptor, and the polymeric Ig receptor for IgA and IgM. The neonatal Fc receptor (FcRn) is a further Fc receptor for IgG, and is involved in IgG transport across epithelial barriers (transcytosis), protecting IgG from degradation, and antigen presentation. Humans have six different classes of Fcγ receptor (mouse orthologues are shown in brackets): FcγRI (mFcγRI), FcγRIIa (mFcγRIII), FcγRIIb (mFcγRIIb), FcγRIIc, FcγRIIIa (mFcγRIV) and FcγRIIIb.

Fcγ receptors may be activatory or inhibitory. Activatory Fcγ receptors FcγRI, FcγRIIa, FcγRIIc and FcγRIIIa comprise immunoreceptor tyrosine-based activation motifs (ITAMs) in their intracellular domains, and ligation by Fc leads to activation of cells expressing the receptors.

The inhibitory Fcγ receptor FcγRIIb comprises immunoreceptor tyrosine-based inhibitory motifs (ITIMs) in its intracellular domain, and negatively regulates cell activation and degranulation, cell proliferation, endocytosis, and phagocytosis upon ligation by Fc.

In this specification an “Fcγ receptor” may be from any species, and includes isoforms, fragments, variants (including mutants) or homologues from any species. Similarly, “FcγRI”, “FcγRIIa”, “FcγRIIb”, “FcγRIIc”, “FcγRIIIa” and “FcγRIIIb” refer respectively to FcγRI/FcγRIIa/FcγRIIb/FcγRIIc/FcγRIIIa/FcγRIIIb from any species, and include isoforms, fragments, variants (including mutants) or homologues from any species. Variant Fcγ receptors include e.g. the 158V and 158F polymorphs of human FcγRIIIa, and the 167H and 167R polymorphs of human FcγRIIa.

In some embodiments, the Fcγ receptor (e.g. FcγRI/FcγRIIa/FcγRIIb/FcγRIIc/FcγRIIIa/FcγRIIIb) is from a mammal (e.g. a primate (rhesus, cynomolgous, non-human primate or human) and/or a rodent (e.g. rat or mouse). Isoforms, fragments, variants or homologues may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature isoform of an Fcγ receptor (e.g. FcγRI/FcγRIIa/FcγRIIb/FcγRIIc/FcγRIIIa/FcγRIIIb) from a given species, e.g. human.

Isoforms, fragments, variants or homologues may optionally be functional isoforms, fragments, variants or homologues, e.g. having a functional property/activity of the reference Fcγ receptor, as determined by analysis by a suitable assay for the functional property/activity. For example, an isoform, fragment, variant or homologue of FcγRI may e.g. display association with human IgG1 Fc.

In this specification an “FcRn receptor” may be from any species, and includes isoforms, fragments, variants (including mutants) or homologues from any species.

In some embodiments, the FcRn receptor is from a mammal (e.g. a primate (rhesus, cynomolgous, non-human primate or human) and/or a rodent (e.g. rat or mouse). Isoforms, fragments, variants or homologues may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature isoform of an FcRn receptor from a given species, e.g. human.

Isoforms, fragments, variants or homologues may optionally be functional isoforms, fragments, variants or homologues, e.g. having a functional property/activity of the reference FcRn, as determined by analysis by a suitable assay for the functional property/activity. For example, an isoform, fragment, variant or homologue of FcRn may e.g. display association with human IgG1 Fc.

The antigen-binding molecule or Fc region of the present invention may be, or may comprise, a complex of polypeptides. The present invention also provides polypeptide constituents of the antigen-binding molecules and Fc regions described herein. The polypeptides may be provided in isolated or substantially purified form.

Linkers and Additional Sequences

In some embodiments the antigen-binding molecules and polypeptides of the present invention comprise one or more linker sequences between amino acid sequences. A linker sequence may be provided at one or both ends of one or more of a VH, VL, CH1-CH2 hinge region, CH2 region and a CH3 region of an antigen-binding molecule/Fc region/polypeptide described herein.

Linker sequences are known to the skilled person, and are described, for example in Chen et al., Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues.

In some embodiments, the linker sequence comprises at least one glycine residue and/or at least one serine residue. In some embodiments the linker sequence consists of glycine and serine residues. In some embodiments, the linker sequence has a length of 1-2, 1-3, 1-4, 1-5, 1-10, 1-15, 1-20, 1-25, or 1-30 amino acids. In some embodiments, the linker sequence comprises, or consists of, an amino sequence comprising one or more (e.g. 1, 2, 3, 4) tandem copies of the amino acid sequence shown in SEQ ID NO:26 or 27.

The antigen-binding molecules and polypeptides of the present invention may additionally comprise further amino acids or sequences of amino acids. For example, the antigen-binding molecules and polypeptides may comprise amino acid sequence(s) to facilitate expression, folding, trafficking, processing, purification or detection of the antigen-binding molecule/polypeptide. For example, the antigen-binding molecule/polypeptide may comprise a sequence encoding a His, (e.g. 6XHis), Myc, GST, MBP, FLAG, HA, E, or Biotin tag, optionally at the N- or C-terminus of the antigen-binding molecule/polypeptide. In some embodiments the antigen-binding molecule/polypeptide comprises a detectable moiety, e.g. a fluorescent, lunminescent, immuno-detectable, radio, chemical, nucleic acid or enzymatic label.

The antigen-binding molecules, Fc regions and polypeptides of the present invention may additionally comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides.

The signal peptide may be present at the N-terminus of the antigen-binding molecule/Fc region/polypeptide, and may be present in the newly synthesised antigen-binding molecule/Fc region/polypeptide. The signal peptide provides for efficient trafficking and secretion of the antigen-binding molecule/Fc region/polypeptide. Signal peptides are often removed by cleavage, and thus are not comprised in the mature antigen-binding molecule/Fc region/polypeptide secreted from the cell expressing the antigen-binding molecule/Fc region/polypeptide.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8: 785-786) or Signal-BLAST (Frank and Sippl, 2008 Bioinformatics 24: 2172-2176).

Labels and Conjugates

In some embodiments the antigen-binding molecules of the present invention additionally comprise a detectable moiety.

In some embodiments the antigen-binding molecule comprises a detectable moiety, e.g. a fluorescent label, phosphorescent label, luminescent label, immuno-detectable label (e.g. an epitope tag), radiolabel, chemical, nucleic acid or enzymatic label. The antigen-binding molecule may be covalently or non-covalently labelled with the detectable moiety.

Fluorescent labels include e.g. fluorescein, rhodamine, allophycocyanin, eosine and NDB, green fluorescent protein (GFP) chelates of rare earths such as europium (Eu), terbium (Tb) and samarium (Sm), tetramethyl rhodamine, Texas Red, 4-methyl umbelliferone, 7-amino-4-methyl coumarin, Cy3, and Cy5. Radiolabels include radioisotopes such as Iodine¹²³, Iodine¹²⁵, Iodine¹²⁶, Iodine¹³¹, Iodine¹³³, Bromine⁷⁷, Technetium^{99m}, Indium¹¹¹, Indium^{113m}, Gallium⁶⁷, Gallium⁶⁸, Ruthenium⁹⁵, Ruthenium⁹⁷, Ruthenium¹⁰³, Ruthenium¹⁰⁵, Mercury²⁰⁷, Mercury²⁰³, Rhenium^{99m}, Rhenium¹⁰¹, Rhenium¹⁰⁵, Scandium⁴⁷, Tellurium^{121m}, Tellurium^{122m}, Tellurium^{125m}, Thulium¹⁶⁵, Thulium¹⁶⁷, Thulium¹⁶⁸, Copper⁶⁷, Fluorine¹⁸, Yttrium⁹⁰, Palladium¹⁰⁰, Bismuth²¹⁷ and Antimony²¹¹. Luminescent labels include as radioluminescent, chemiluminescent (e.g. acridinium ester, luminol, isoluminol) and bioluminescent labels. Immuno-detectable labels include haptens, peptides/polypeptides, antibodies, receptors and ligands such as biotin, avidin, streptavidin or digoxigenin. Nucleic acid labels include aptamers. Enzymatic labels include e.g. peroxidase, alkaline phosphatase, glucose oxidase, beta-galactosidase and luciferase. In some embodiments the antigen-binding molecules of the present invention are conjugated to a chemical moiety. The chemical moiety may be a moiety for providing a therapeutic effect. Antibody-drug conjugates are reviewed e.g. in Parslow et al., Biomedicines. 2016 September; 4(3):14. In some embodiments, the chemical moiety may be a drug moiety (e.g. a cytotoxic agent). In some embodiments, the drug moiety may be a chemotherapeutic agent. In some embodiments, the drug moiety is selected from calicheamicin, DM1, DM4, monomethylauristatin E (MMAE), monomethylauristatin F (MMAF), SN-38, doxorubicin, duocarmycin, D6.5 and PBD.

Functional Properties

The antigen-binding molecules, Fc regions and polypeptides described herein may be characterised by reference to certain functional properties.

In some embodiments, an antigen-binding molecule comprising an Fc region as described herein may possess one or more of the following properties:
Binds to an activatory Fcγ receptor (e.g. hFcγRIIa (e.g. hFcγRIIa167H, hFcγRIIa167R), hFcγRIIIa (e.g. hFcγRIIIa158V, hFcγRIIIa158F), mFcγRIV, mFcγRIII);
Binds to FcRn (e.g. hFcRn, mFcRn);

Increased binding to an activatory Fcγ receptor (e.g. hFcγRIIa (e.g. hFcγRIIa167H, hFc1γRIIa167R), hFcγRIIIa (e.g. hFcγRIIIa158V, hFcγRIIIa158F), mFcγRIV, mFcγRIII) as compared to an equivalent antigen-binding molecule having an Fc region comprised of CH2-CH3 having the amino acid sequence of SEQ ID NO:6 or 31;
Increased binding to FcRn (e.g. hFcRn, mFcRn) as compared to an equivalent antigen-binding molecule having an Fc region comprised of CH2-CH3 having the amino acid sequence of SEQ ID NO:6 or 31;
Decreased binding to an inhibitory Fcγ receptor (e.g. hFcγRIIb, mFcγRIIb) as compared to an equivalent antigen-binding molecule having an Fc region comprised of CH2-CH3 having the amino acid sequence of SEQ ID NO:6 or 31;
Increased binding to, and/or selectivity for, an activatory Fcγ receptor over an inhibitory Fcγ receptor (e.g. increased selectivity for hFcγRIIa over hFcγRIIb) as compared to an equivalent antigen-binding molecule having an Fc region comprised of CH2-CH3 having the amino acid sequence of SEQ ID NO:6 or 31;
Increased or decreased binding to a complement protein (e.g. C1q) as compared to an equivalent antigen-binding molecule having an Fc region comprised of CH2-CH3 having the amino acid sequence of SEQ ID NO:6 or 31;
Increased hexamerisation as compared to an equivalent antigen-binding molecule having an Fc region comprised of CH2-CH3 having the amino acid sequence of SEQ ID NO:6 or 31;
Increased ADCC activity as compared to an equivalent antigen-binding molecule having an Fc region comprised of CH2-CH3 having the amino acid sequence of SEQ ID NO:6 or 31;
Increased ADCP activity as compared to an equivalent antigen-binding molecule having an Fc region comprised of CH2-CH3 having the amino acid sequence of SEQ ID NO:6 or 31;
Increased CDC activity as compared to an equivalent antigen-binding molecule having an Fc region comprised of CH2-CH3 having the amino acid sequence of SEQ ID NO:6 or 31;
Similar or increased thermostability as compared to an equivalent antigen-binding molecule having an Fc region comprised of CH2-CH3 having the amino acid sequence of SEQ ID NO:6 or 31, or
Increased inhibition of tumor growth in vivo as compared to an equivalent antigen-binding molecule having an Fc region comprised of CH2-CH3 having the amino acid sequence of SEQ ID NO:6 or 31.

Binding to Fc receptors can be analysed by methods well known to the skilled person, including e.g. ELISA, Surface Plasmon Resonance (SPR; see e.g. Hearty et al., Methods Mol Biol (2012) 907:411-442), Bio-Layer Interferometry (see e.g. Lad et al., (2015) J Biomol Screen 20(4): 498-507). The affinity of binding may be determined, and may e.g. be expressed as K_{D} value.

Selectivity of binding to a given Fc receptor as compared to another can be determined e.g. by determining the affinity of binding to each receptor. Selectivity of binding can be expressed as the fraction of the K_{D} values for binding to different Fc receptors.

ADCC activity can be analysed e.g. according to the methods described in Yamashita et al., Scientific Reports (2016) 6:19772 (hereby incorporated by reference in its entirety), or by ⁵¹Cr release assay as described e.g. in Jedema et al., Blood (2004) 103: 2677-82 (hereby incorporated by reference in its entirety). ADCC activity can also be analysed using the Pierce LDH Cytotoxicity Assay Kit, in accordance with the manufacturer's instructions (as described in Example 5 herein).

ADCP can be analysed e.g. according to the method described in Kamen et al., J Immunol (2017) 198 (1 Supplement) 157.17 (hereby incorporated by reference in its entirety).

The ability to induce CDC can be analysed e.g. using a C1q binding assay, e.g. as described in Schlothauer et al., Protein Engineering, Design and Selection (2016), 29(10):457-466 (hereby incorporated by reference in its entirety).

Thermostability of antigen-binding molecules can be analysed by methods well known to the skilled person, including Differential Scanning Fuorimetry and Differential Scanning calorimetry (DSC), which are described e.g. in He et al., J Pharm Sci. (2010) which is hereby incorporated by reference in its entirety. Thermostability may be reflected in terms of a melting temperature (Tₘ), unfolding temperature or disassembly temperature (expressed e.g. in ° C. or ° F.

Tumor growth inhibition can be analysed in an appropriate in vivo model, e.g. cell line-derived xenograft model such as A549 cell-derived model.

In some embodiments, an antigen-binding molecule comprising an Fc region as described herein binds to an activatory Fcγ receptor (e.g. hFcγRIIa (e.g. hFcγRIIa167H, hFcγRIIa167R), hFcγRIIIa (e.g. hFcγRIIIa158V, hFcγRIIIa158F), mFcγRIV, mFcγRIII) with an affinity of binding which is greater than 1 times, e.g. greater than 2, 3, 4, 5, 6, 7, 8, 9, 10, 15, or greater than 20 times the affinity of binding to the activatory Fcγ receptor by an equivalent antigen-binding molecule having an Fc region comprised of CH2-CH3 having the amino acid sequence of SEQ ID NO:6 or 31. In some embodiments the K_{D} of the antigen-binding molecule comprising an Fc region described herein for binding to the activatory Fcγ receptor is less than 1 times, e.g. less than 0.9, 0.8, 0.7, 0.6, 0.5, 0.4, 0.3, 0.2, 0.1, 0.09, 0.08, 0.07, 0.06 or less than 0.05 times the K_{D} of an equivalent antigen-binding molecule having an Fc region comprised of CH2-CH3 having the amino acid sequence of SEQ ID NO:6 or 31 for the activatory Fcγ receptor.

In some embodiments, the antigen-binding molecule comprising an Fc region as described herein binds to an activatory Fcγ receptor (e.g. hFcγRIIa (e.g. hFcγRIIa167H, hFcγRIIa167R), hFcγRIIIa (e.g. hFcγRIIIa158V, hFcγRIIIa158F), mFcγRIV, mFcγRIII) with a K_{D} of 1000 nM or less, preferably one of ≤500 nM, ≤100nM, ≤75nM, ≤50nM, ≤40 nM, ≤30nM, ≤20nM, ≤15 nM, ≤12.5 nM, ≤10 nM, ≤9 nM, ≤8 nM, ≤7 nM, ≤6 nM, ≤5 nM, ≤4 nM, ≤3 nM, ≤2 nM or ≤1 nM.

In some embodiments, an antigen-binding molecule comprising an Fc region as described herein binds to an FcRn (e.g. hFcRn, mFcRn) with an affinity of binding which is greater than 1 times, e.g. greater than 2, 3, 4, 5, 6, 7, 8, 9, 10, 15, or greater than 20 times the affinity of binding to the FcRn by an equivalent antigen-binding molecule having an Fc region comprised of CH2-CH3 having the amino acid sequence of SEQ ID NO:6 or 31. In some embodiments the K_{D} of the antigen-binding molecule comprising an Fc region described herein for binding to the FcRn is less than 1 times, e.g. less than 0.9, 0.8, 0.7, 0.6, 0.5, 0.4, 0.3, 0.2, 0.1, 0.09, 0.08, 0.07, 0.06 or less than 0.05 times the K_{D} of an equivalent antigen-binding molecule having an Fc region comprised of CH2-CH3 having the amino acid sequence of SEQ ID NO:6 or 31 for the FcRn.

In some embodiments, the antigen-binding molecule comprising an Fc region as described herein binds to an FcRn (e.g. hFcRn, mFcRn) with a K_{D} of 1000 nM or less, preferably one of ≤500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM, ≤3 nM, ≤2 nM or ≤1 nM.

In some embodiments, an antigen-binding molecule comprising an Fc region as described herein binds to an inhibitory Fcγ receptor (e.g. hFcγRIIb mFcγRIIb) with an affinity of binding which is less than 1 times, e.g. less than 0.9, 0.8, 0.7, 0.6, 0.5, 0.4, 0.3, 0.2, or less than 0.1 times the affinity of binding to the inhibitory Fcγ receptor by an equivalent antigen-binding molecule having an Fc region comprised of CH2-CH3 having the amino acid sequence of SEQ ID NO:6 or 31. In some embodiments the K_{D} of the antigen-binding molecule comprising an Fc region described herein for binding to the inhibitory Fcγ receptor is greater than 1 times, e.g. greater than 2, 3, 4, 5, 6, 7, 8, 9 or greater than 10 times the K_{D} of an equivalent antigen-binding molecule having an Fc region comprised of CH2-CH3 having the amino acid sequence of SEQ ID NO:6 or 31 for the inhibitory Fcγ receptor.

In some embodiments, the antigen-binding molecule comprising an Fc region as described herein binds to an inhibitory Fcγ receptor (e.g. hFcγRIIb mFcγRIIb) with a K_{D} 1 nM or greater, preferably one of ≥5 nM, ≥10 nM, ≥50 nM, ≥100 nM, ≥500 nM, ≥1000 nM, ≥2000 nM, ≥3000 nM, ≥4000 nM or ≥5000 nM.

In some embodiments the selectivity of binding for an activatory Fcγ receptor (e.g. hFcγRIIa) relative to an inhibitory Fcγ receptor (e.g. hFcγRIIb) for an antigen-binding molecule comprising an Fc region as described herein is greater than 1 times, e.g. greater than 2, 3, 4, 5, 6, 7, 8, 9, 10, 15, or greater than 20 times selectivity of binding displayed by an equivalent antigen-binding molecule having an Fc region comprised of CH2-CH3 having the amino acid sequence of SEQ ID NO:6 or 31.

In some embodiments, an antigen-binding molecule comprising an Fc region as described herein displays ADCC which is greater than 1 times, e.g. greater than 2, 3, 4, 5, 6, 7, 8, 9, 10, 15, or greater than 20 times the ADCC displayed by an equivalent antigen-binding molecule having an Fc region comprised of CH2-CH3 having the amino acid sequence of SEQ ID NO:6 or 31.

In some embodiments, the EC50 (ng/ml) determined for an antigen-binding molecule comprising an Fc region as described herein in an assay of ADCC activity less than 1 times, e.g. less than 0.9, 0.8, 0.7, 0.6, 0.5, 0.4, 0.3, 0.2, or less than 0.1 times the EC50 (ng/ml) determined for an equivalent antigen-binding molecule having an Fc region comprised of CH2-CH3 having the amino acid sequence of SEQ ID NO:6 or 31.

In some embodiments, the EC50 (ng/ml) for an antigen-binding molecule comprising an Fc region as described herein in an assay of ADCC activity is 500 ng/ml or less, preferably one of ≤400 ng/ml, ≤300 ng/ml, ≤200 ng/ml, ≤100 ng/ml, ≤90 ng/ml, ≤80 ng/ml, ≤70 ng/ml, ≤60 ng/ml, ≤50 ng/ml, ≤40 ng/ml, ≤30 ng/ml, ≤20 ng/ml, or ≤10 ng/ml.

In some embodiments, an antigen-binding molecule comprising an Fc region as described herein may have a melting temperature, unfolding temperature or disassembly temperature which is which is ≥0.75 times and ≤1.25 times, e.g. ≥0.8 times and ≤1.2 times, ≥0.85 times and ≤1.15 times, ≥0.9 times and ≤1.1 times, ≥0.91 times and ≤1.09 times, ≥0.92 times and ≤1.08 times, ≥0.93 times and ≤1.07 times, ≥0.94 times and ≤1.06 times, ≥0.95 times and ≤1.05 times, ≥0.96 times and ≤1.04 times, ≥0.97 times and ≤1.03 times, ≥0.98 times and ≤1.02 times, or ≥0.99 times and ≤1.01 times the melting temperature, unfolding temperature or disassembly temperature of an equivalent antigen-binding molecule having an Fc region comprised of CH2-CH3 having the amino acid sequence of SEQ ID NO:6 or 31.

In some embodiments, an antigen-binding molecule comprising an Fc region as described herein may inhibit tumor growth in vivo to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of tumor growth inhibition observed for an equivalent antigen-binding molecule having an Fc region comprised of CH2-CH3 having the amino acid sequence of SEQ ID NO:6 or 31.

Nucleic Acids and Vectors

The present invention provides a nucleic acid, or a plurality of nucleic acids, encoding an antigen-binding molecule, Fc region or polypeptide according to the present invention.

In some embodiments, the nucleic acid is purified or isolated, e.g. from other nucleic acid, or naturally-occurring biological material. In some embodiments the nucleic acid(s) comprise or consist of DNA and/or RNA.

The present invention also provides a vector, or plurality of vectors, comprising the nucleic acid or plurality of nucleic acids according to the present invention.

The nucleotide sequence may be contained in a vector, e.g. an expression vector. A “vector” as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express a peptide or polypeptide from a vector according to the invention.

The term “operably linked” may include the situation where a selected nucleic acid sequence and regulatory nucleic acid sequence (e.g. promoter and/or enhancer) are covalently linked in such a way as to place the expression of nucleic acid sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. The resulting transcript(s) may then be translated into a desired peptide(s)/polypeptide(s).

Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes).

In some embodiments, the vector may be a eukaryotic vector, e.g. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression.

Constituent polypeptides of an antigen-binding molecule or Fc region according to the present invention may be encoded by different nucleic acids of the plurality of nucleic acids, or by different vectors of the plurality of vectors.

Cells Comprising/Expressing the Antigen-Binding Molecules and Polypeptides

The present invention also provides a cell comprising or expressing an antigen-binding molecule, Fc region or polypeptide according to the present invention. Also provided is a cell comprising or expressing a nucleic acid, a plurality of nucleic acids, a vector or a plurality of vectors according to the invention.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

The present invention also provides a method for producing a cell comprising a nucleic acid(s) or vector(s) according to the present invention, comprising introducing a nucleic acid, a plurality of nucleic acids, a vector or a plurality of vectors according to the present invention into a cell. In some embodiments, introducing an isolated nucleic acid(s) or vector(s) according to the invention into a cell comprises transformation, transfection, electroporation or transduction (e.g. retroviral transduction).

The present invention also provides a method for producing a cell expressing/comprising an antigen-binding molecule, Fc region or polypeptide according to the present invention, comprising introducing a nucleic acid, a plurality of nucleic acids, a vector or a plurality of vectors according to the present invention in a cell. In some embodiments, the methods additionally comprise culturing the cell under conditions suitable for expression of the nucleic acid(s) or vector(s) by the cell. In some embodiments, the methods are performed in vitro.

The present invention also provides cells obtained or obtainable by the methods according to the present invention.

Producing the Antigen-Binding Molecules, Fc Regions and Polypeptides

Antigen-binding molecules, Fc regions and polypeptides according to the invention may be prepared according to methods for the production of polypeptides known to the skilled person.

Polypeptides may be prepared by chemical synthesis, e.g. liquid or solid phase synthesis. For example, peptides/polypeptides can by synthesised using the methods described in, for example, Chandrudu et al., Molecules (2013), 18: 4373-4388, which is hereby incorporated by reference in its entirety.

Alternatively, antigen-binding molecules, Fc regions and polypeptides may be produced by recombinant expression. Molecular biology techniques suitable for recombinant production of polypeptides are well known in the art, such as those set out in Green and Sambrook, Molecular Cloning: A Laboratory Manual (4th Edition), Cold Spring Harbor Press, 2012, and in Nat Methods. (2008); 5(2): 135-146 both of which are hereby incorporated by reference in their entirety. Methods for the recombinant production of antigen-binding molecules are also described in Frenzel et al., Front Immunol. (2013); 4: 217 and Kunert and Reinhart, Appl Microbiol Biotechnol. (2016) 100: 3451-3461, both of which are hereby incorporated by reference in their entirety.

In some cases the antigen-binding molecules and Fc regions of the present invention are comprised of more than one polypeptide chain. In such cases, production may comprise transcription and translation of more than one polypeptide, and subsequent association of the polypeptide chains to form the antigen-binding molecule/Fc region.

For recombinant production according to the invention, any cell suitable for the expression of polypeptides may be used. The cell may be a prokaryote or eukaryote. In some embodiments the cell is a prokaryotic cell, such as a cell of archaea or bacteria. In some embodiments the bacteria may be Gram-negative bacteria such as bacteria of the family Enterobacteriaceae, for example Escherichia coli. In some embodiments, the cell is a eukaryotic cell such as a yeast cell, a plant cell, insect cell or a mammalian cell, e.g. CHO, HEK (e.g. HEK293), HeLa or COS cells.

In some cases the cell is not a prokaryotic cell because some prokaryotic cells do not allow for the same folding or post-translational modifications as eukaryotic cells. In addition, very high expression levels are possible in eukaryotes and proteins can be easier to purify from eukaryotes using appropriate tags. Specific plasmids may also be utilised which enhance secretion of the protein into the media.

In some embodiments polypeptides may be prepared by cell-free-protein synthesis (CFPS), e.g. according using a system described in Zemella et al. Chembiochem (2015) 16(17): 2420-2431, which is hereby incorporated by reference in its entirety.

Production may involve culture or fermentation of a eukaryotic cell modified to express the polypeptide(s) of interest. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide(s). Culture, fermentation and separation techniques are well known to those of skill in the art, and are described, for example, in Green and Sambrook, Molecular Cloning: A Laboratory Manual (4th Edition; incorporated by reference herein above).

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culturing the cells that express the antigen-binding molecule/Fc region/polypeptide(s), the polypeptide(s) of interest may be isolated. Any suitable method for separating proteins from cells known in the art may be used. In order to isolate the polypeptide it may be necessary to separate the cells from nutrient medium. If the polypeptide(s) are secreted from the cells, the cells may be separated by centrifugation from the culture media that contains the secreted polypeptide(s) of interest. If the polypeptide(s) of interest collect within the cell, protein isolation may comprise centrifugation to separate cells from cell culture medium, treatment of the cell pellet with a lysis buffer, and cell disruption e.g. by sonification, rapid freeze-thaw or osmotic lysis.

It may then be desirable to isolate the polypeptide(s) of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size chromatography. These may be used as an alternative to precipitation, or may be performed subsequently to precipitation.

Once the polypeptide(s) of interest have been isolated from culture it may be desired or necessary to concentrate the polypeptide(s). A number of methods for concentrating proteins are known in the art, such as ultrafiltration or lyophilisation.

Compositions

The present invention also provides compositions comprising the antigen-binding molecules, Fc regions, polypeptides, nucleic acids, expression vectors and cells described herein.

The antigen-binding molecules, Fc regions, polypeptides, nucleic acids, expression vectors and cells described herein may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion.

Suitable formulations may comprise the antigen-binding molecule in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments the composition is formulated for injection or infusion, e.g. into a blood vessel or tumor.

In accordance with the invention described herein methods are also provided for the production of pharmaceutically useful compositions, such methods of production may comprise one or more steps selected from: producing an antigen-binding molecule, Fc region, polypeptide, nucleic acid (or plurality thereof), expression vector (or plurality thereof) or cell described herein; isolating an antigen-binding molecule, Fc region, polypeptide, nucleic acid (or plurality thereof), expression vector (or plurality thereof) or cell described herein; and/or mixing antigen-binding molecule, Fc region polypeptide, nucleic acid (or plurality thereof), expression vector (or plurality thereof) or cell described herein with a pharmaceutically acceptable carrier, adjuvant, excipient or diluent.

For example, a further aspect the invention described herein relates to a method of formulating or producing a medicament or pharmaceutical composition for use in the treatment of a disease/condition (e.g. a cancer), the method comprising formulating a pharmaceutical composition or medicament by mixing an antigen-binding molecule, Fc region, polypeptide, nucleic acid (or plurality thereof), expression vector (or plurality thereof) or cell described herein with a pharmaceutically acceptable carrier, adjuvant, excipient or diluent.

Methods Using the Articles of the Present Disclosure

The articles of the present disclosure are useful in methods employing Fc effector function.

In particular, the antigen-binding molecules, Fc regions, polypeptides, nucleic acids, expression vectors, cells and compositions described herein are useful in methods employing an Fc-mediated effector function such as antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, or antigen processing and presentation.

Such methods include: methods for killing cells expressing a target antigen, methods for reducing the number of cells expressing a target antigen, methods for inhibiting the activity of cells expressing a target antigen, methods for enhancing an immune response against cells expressing a target antigen, methods for promoting the lysis of cells expressing a target antigen, methods for increasing phagocytosis of cells expressing a target antigen.

The methods may comprise contacting a cell or cells expressing a target antigen with an antigen-binding molecule, cell or composition described herein. In some embodiments methods further comprise contacting cells expressing a target antigen with effector cells for the relevant activity (e.g. NK cells, macrophages, eosinophils, neutrophils (for ADCC), phagocytic cells (monocytes, macrophages, neutrophils, tissue dendritic cells, mast cells). The methods may be in vitro, in vivo or ex vivo methods.

The antigen-binding molecules, nucleic acids, expression vectors, cells and compositions described herein find use in therapeutic and prophylactic methods.

The present invention provides an antigen-binding molecule, nucleic acid (or plurality thereof), expression vector (or plurality thereof), cell or composition described herein for use in a method of medical treatment or prophylaxis. Also provided is the use of an antigen-binding molecule, nucleic acid (or plurality thereof), expression vector (or plurality thereof), cell or composition described herein in the manufacture of a medicament for treating or preventing a disease or condition. Also provided is a method of treating or preventing a disease or condition, comprising administering to a subject a therapeutically or prophylactically effective amount of an antigen-binding molecule, nucleic acid (or plurality thereof), expression vector (or plurality thereof), cell or composition described herein.

The methods may be effective to reduce the development or progression of a disease/condition, alleviation of the symptoms of a disease/condition or reduction in the pathology of a disease/condition. The methods may be effective to prevent progression of the disease/condition, e.g. to prevent worsening of, or to slow the rate of development of, the disease/condition. In some embodiments the methods may lead to an improvement in the disease/condition, e.g. a reduction in the symptoms of the disease/condition or reduction in some other correlate of the severity/activity of the disease/condition. In some embodiments the methods may prevent development of the disease/condition a later stage (e.g. a chronic stage or metastasis).

It will be appreciated that the articles of the present invention may be used for the treatment/prevention of any disease/condition that would derive therapeutic or prophylactic benefit from a reduction in the number and/or activity of cells expressing the target antigen. For example, the disease/condition may be a disease/condition in which cells expressing the target antigen are pathologically implicated, e.g. a disease/condition in which an increase in the level of the target antigen, or an increased number/proportion of cells expressing the target antigen, is positively associated with the onset, development or progression of the disease/condition, and/or severity of one or more symptoms of the disease/condition, or for which increase in the level of the target antigen, or an increased number/proportion of cells expressing the target antigen, is a risk factor for the onset, development or progression of the disease/condition.

In some embodiments, the disease/condition to be treated/prevented is a disease/condition characterised by an increase in the number/proportion/activity of cells expressing the target antigen, e.g. as compared to the number/proportion/activity of cells expressing the target antigen in the absence of the disease/condition.

In some embodiments the disease/condition to be treated/prevented is a cancer. The cancer may be any unwanted cell proliferation (or any disease manifesting itself by unwanted cell proliferation), neoplasm or tumor. The cancer may be benign or malignant and may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, hematologic cancer and sarcoma.

The treatment/prevention may be aimed at one or more of: delaying/preventing the onset/progression of symptoms of the cancer, reducing the severity of symptoms of the cancer, reducing the survival/growth/invasion/metastasis of cells of the cancer, reducing the number of cells of the cancer and/or increasing survival of the subject.

In some embodiments, the cancer to be treated/prevented comprises cells expressing the target antigen. In some embodiments, the cancer to be treated/prevented is a cancer which is positive for the target antigen. In some embodiments, the cancer over-expresses the target antigen. Overexpression of the target antigen can be determined by detection of a level of expression of the target antigen which is greater than the level of expression by equivalent non-cancerous cells/non-tumor tissue.

The target antigen expression may be determined by any suitable means. Expression may be gene expression or protein expression. Gene expression can be determined e.g. by detection of mRNA encoding the target antigen, for example by quantitative real-time PCR (qRT-PCR). Protein expression can be determined e.g. by detection of the target antigen, for example by antibody-based methods, for example by western blot, immunohistochemistry, immunocytochemistry, flow cytometry, or ELISA.

In some embodiments, a patient may be selected for treatment described herein based on the detection of a cancer expressing the target antigen, or overexpressing the target antigen, e.g. in a sample obtained from the subject.

In some embodiments the cancer to be treated/prevented in accordance with the present invention is selected from: a solid cancer, a liquid cancer, gastric cancer (e.g. gastric carcinoma, gastric adenocarcinoma, gastrointestinal adenocarcinoma), liver cancer (hepatocellular carcinoma, cholangiocarcinoma), head and neck cancer (e.g. head and neck squamous cell carcinoma), breast cancer, ovarian cancer (e.g. ovarian carcinoma), lung cancer (e.g. NSCLC, lung adenocarcinoma, squamous lung cell carcinoma), brain cancer (e.g. glioblastoma) skin cancer (e.g. melanoma), prostate cancer, oral cavity cancer (e.g. oropharyngeal cancer), renal cancer (e.g. renal cell carcinoma) colorectal cancer (e.g. colorectal carcinoma), oesophageal cancer, pancreatic cancer, bladder cancer, a hematologic malignancy, a myeloid hematologic malignancy, a lymphoblastic hematologic malignancy, myelodysplastic syndrome (MDS), acute myeloid leukemia (AML), chronic myeloid leukemia (CML), acute lymphoblastic leukemia (ALL), lymphoma, non-Hodgkin's lymphoma (NHL), thymoma or multiple myeloma (MM).

In some embodiments the disease/condition is an infectious disease (i.e. a disease/condition caused by an infectious agent). In such embodiments the treatment may be aimed at reducing the number of infected cells (e.g. in the case of infectious disease associated with infection by an intracellular pathogen) and/or killing the infectious agent.

An infection may be any infection or infectious disease, e.g. viral, bacterial, fungal, or parasitic infection. In some embodiments, the disease/disorder may be associated with infection by an intracellular pathogen. In some embodiments, the disease/disorder may be associated with infection by a virus. In some embodiments it may be particularly desirable to treat chronic/persistent infections. The infection may be chronic, persistent, latent or slow, and may be the result of viral, bacterial, fungal or parasitic infection. As such, treatment may be provided to patients having a bacterial, viral or fungal infection.

Examples of bacterial infections that may be treated include infection by Bacillus spp., Bordetella pertussis, Clostridium spp., Corynebacterium spp., Vibrio cholerae, Staphylococcus spp., Streptococcus spp. Escherichia, Klebsiella, Proteus, Yersinia, Erwinia, Salmonella, Listeria sp, Helicobacter pylori, mycobacteria (e.g. Mycobacterium tuberculosis) and Pseudomonas aeruginosa. For example, the bacterial infection may be sepsis or tuberculosis. Examples of viral infections that may be treated include infection by influenza virus, measles virus, hepatitis B virus (HBV), hepatitis C virus (HCV), human immunodeficiency virus (HIV), lymphocytic choriomeningitis virus (LCMV), Herpes simplex virus and human papilloma virus (HPV). Examples of fungal infections that may be treated include infection by Alternaria sp, Aspergillus sp, Candida sp and Histoplasma sp. The fungal infection may be fungal sepsis or histoplasmosis. Examples of parasitic infections that may be treated include infection by Plasmodium species (e.g. Plasmodium falciparum, Plasmodium yoeli, Plasmodium ovale, Plasmodium vivax, or Plasmodium chabaudi chabaudi). The parasitic infection may be a disease such as malaria, leishmaniasis and toxoplasmosis.

In some embodiments the disease/condition is an autoimmune disease. In some embodiments the autoimmune disease is selected from: diabetes mellitus type 1, celiac disease, Graves' disease, inflammatory bowel disease, multiple sclerosis, psoriasis, rheumatoid arthritis, and systemic lupus erythematosus.

Administration of the articles of the present invention is preferably in a “therapeutically effective” or “prophylactically effective” amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Administration may be alone or in combination with other treatments, either simultaneously or sequentially dependent upon the condition to be treated. The antigen-binding molecule or composition described herein and a therapeutic agent may be administered simultaneously or sequentially.

Simultaneous administration refers to administration of an article of the present invention and another therapeutic agent together, for example as a pharmaceutical composition containing both agents (combined preparation), or immediately after each other and optionally via the same route of administration, e.g. to the same artery, vein or other blood vessel. Sequential administration refers to administration of one agent followed after a given time interval by separate administration of the other agent. It is not required that the two agents are administered by the same route, although this is the case in some embodiments. The time interval may be any time interval.

Chemotherapy and radiotherapy respectively refer to treatment of a cancer with a drug or with ionising radiation (e.g. radiotherapy using X-rays or y-rays). The drug may be a chemical entity, e.g. small molecule pharmaceutical, antibiotic, DNA intercalator, protein inhibitor (e.g. kinase inhibitor), or a biological agent, e.g. antibody, antibody fragment, aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat a cancer.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment. The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs may be selected from: Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (lmiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Ciloleucel, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin) , Bevacizumab, Bexarotene, Bexxar (Tositumomab and Iodine 1131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calquence (Acalabrutinib), Campath (Alemtuzumab), Camptosar (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine 1131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), Vel P, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib) and Zytiga (Abiraterone Acetate).

In some embodiments the treatment may comprise administration of a corticosteroid, e.g. dexamethasone and/or prednisone.

Multiple doses of the producing an antigen-binding molecule, polypeptide, CAR, nucleic acid (or plurality thereof), expression vector (or plurality thereof), cell or composition may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

Subjects

The subject in accordance with aspects the invention described herein may be any animal or human. The subject is preferably mammalian, more preferably human. The subject may be a non-human mammal, but is more preferably human. The subject may be male or female. The subject may be a patient. A subject may have been diagnosed with a disease or condition requiring treatment (e.g. a cancer), may be suspected of having such a disease/condition, or may be at risk of developing/contracting such a disease/condition.

In embodiments according to the present invention the subject is preferably a human subject. In some embodiments, the subject to be treated according to a therapeutic or prophylactic method of the invention herein is a subject having, or at risk of developing, a cancer. In embodiments according to the present invention, a subject may be selected for treatment according to the methods based on characterisation for certain markers of such disease/condition.

Kits

In some aspects of the invention described herein a kit of parts is provided. In some embodiments the kit may have at least one container having a predetermined quantity of an antigen-binding molecule, Fc region, polypeptide, nucleic acid (or plurality thereof), expression vector (or plurality thereof), cell or composition described herein.

In some embodiments, the kit may comprise materials for producing an antigen-binding molecule, Fc region, polypeptide, nucleic acid (or plurality thereof), expression vector (or plurality thereof), cell or composition described herein.

The kit may provide the antigen-binding molecule, Fc region, polypeptide, nucleic acid (or plurality thereof), expression vector (or plurality thereof), cell or composition together with instructions for administration to a patient in order to treat a specified disease/condition.

In some embodiments the kit may further comprise at least one container having a predetermined quantity of another therapeutic agent (e.g. anti-infective agent or chemotherapy agent). In such embodiments, the kit may also comprise a second medicament or pharmaceutical composition such that the two medicaments or pharmaceutical compositions may be administered simultaneously or separately such that they provide a combined treatment for the specific disease or condition. The therapeutic agent may also be formulated so as to be suitable for injection or infusion to a tumor or to the blood.

Sequence Identity

As used herein, “sequence identity” refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Soding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

Sequences

| SEQ ID | | |
|---|---|---|
| NO: | DESCRIPTION | SEQUENCE |
| 1 | Human IgG1 G1m1 allotype | ASTKGPSVFPLAPSSKSTSGGTAALGCLVKDYFPEPVTVSWNSGALTSGVHTFPAVLQ |
| | constant region (IGHG1; | SSGLYSLSSVVTVPSSSLGTQTYICNVNHKPSNTKVDKKVEPKSCDKTHTCPPCPAPE |
| | UniProt: P01857-1, v1) | LLGGPSVFLFPPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 2 | CH1 IgG1 (positions 1-98 of | ASTKGPSVFPLAPSSKSTSGGTAALGCLVKDYFPEPVTVSWNSGALTSGVHTFPAVLQ |
| | P01857-1, v1) | SSGLYSLSSVVTVPSSSLGTQTYICNVNHKPSNTKVDKKV |
| 3 | Hinge IgG1 (positions 99-110 | EPKSCDKTHTCP |
| | of P01857-1, v1) | |
| 4 | CH2 IgG1 (positions 111-223 | PCPAPELLGGPSVFLFPPKPKDTLMISRTPEVTCVVVDVSHEDPEVKFNWYVDGVEVH |
| | of P01857-1, v1) | NAKTKPREEQYNSTYRVVSVLTVLHQDWLNGKEYKCKVSNKALPAPIEKTISKAK |
| 5 | CH3 IgG1 (positions 224-330 | GQPREPQVYTLPPSRDELTKNQVSLTCLVKGFYPSDIAVEWESNGQPENNYKTTPPVL |
| | of P01857-1, v1) | DSDGSFFLYSKLTVDKSRWQQGNVFSCSVMHEALHNHYTQKSLSLSPGK |
| 6 | CH2-CH3 IgG1 | PCPAPELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKGQP |
| | | REPQVYTLPPSRDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| 7 | C_{K} CL (IGCK; UniProt: | RTVAAPSVFIFPPSDEQLKSGTASVVCLLNNFYPREAKVQWKVDNALQSGNSQESVTE |
| | P01834-1, v2) | QDSKDSTYSLSSTLTLSKADYEKHKVYACEVTHQGLSSPVTKSFNRGEC |
| 8 | CH2_GASD | PCPAPELLAGPDVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAK |
| 9 | CH2_GASDIE | PCPAPELLAGPDVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPEEKTISKAK |
| 10 | CH2_GASDALIE | PCPAPELLAGPDVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPLPEEKTISKAK |
| 11 | CH2_LCKC | PCPAPELLGGPSVFCFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPIECTISKAK |
| 12 | CH2_GASDLCKC | PCPAPELLAGPDVFCFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIECTISKAK |
| 13 | CH2_GASDIELCKC | PCPAPELLAGPDVFCFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPEECTISKAK |
| 14 | CH2_GASDALIELCKC | PCPAPELLAGPDVFCFPPKPKDTLMISRTPEVTCVVVDVSHEDPEVKFNWYVDGVEVH |
| | | NAKTKPREEQYNSTYRVVSVLTVLHQDWLNGKEYKCKVSNKALPLPEECTISKAK |
| 15 | CH3_EKEG | GQPRKPQVYTLPPSRDELTKNQVSLTCLVKGFYPSDIAVEWESNGQPENNYKTTPPVL |
| | | DSDGSFFLYSKLTVDKSRWQQGNVFSCSVMHGALHNHYTQKSLSLSPGK |
| 16 | CH2-CH3_GASD | PCPAPELLAGPDVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKGQP |
| | | REPQVYTLPPSRDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| 17 | CH2-CH3_GASDIE | PCPAPELLAGPDVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPEEKTISKAKGQP |
| | | REPQVYTLPPSRDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| 18 | CH2-CH3_GASDALIE | PCPAPELLAGPDVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPLPEEKTISKAKGQP |
| | | REPQVYTLPPSRDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| 19 | CH2-CH3_LCKC | PCPAPELLGGPSVFCFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPIECTISKAKGQ |
| | | PREPQVYTLPPSRDELTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |
| 20 | CH2-CH3_GASDLCKC | PCPAPELLAGPDVFCFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIECTISKAKGQP |
| | | REPQVYTLPPSRDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| 21 | CH2-CH3_GASDIELCKC | PCPAPELLAGPDVFCFPPKPKDTLMISRTPEVTCVVVDVSHEDPEVKFNVVYVDGVEVH |
| | | NAKTKPREEQYNSTYRVVSVLTVLHQDWLNGKEYKCKVSNKALPAPEECTISKAKGQ |
| | | PREPQVYTLPPSRDELTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |
| 22 | CH2-CH3_GASDALIELCKC | PCPAPELLAGPDVFCFPPKPKDTLMISRTPEVTCVVVDVSHEDPEVKFNWYVDGVEVH |
| | | NAKTKPREEQYNSTYRVVSVLTVLHQDWLNGKEYKCKVSNKALPLPEECTISKAKGQP |
| | | REPQVYTLPPSRDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| 23 | CH2-CH3_EKEG | PCPAPELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKGQP |
| | | RKPQVYTLPPSRDELTKNQVSLTCLVKGFYPSDIAVEWESNGQPENNYKTTPPVLDSD |
| | | GSFFLYSKLTVDKSRWQQGNVFSCSVMHGALHNHYTQKSLSLSPGK |
| 24 | CH2-CH3_LCKCEKEG | PCPAPELLGGPSVFCFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPIECTISKAKGQ |
| | | PRKPQVYTLPPSRDELTKNQVSLTCLVKGFYPSDIAVEWESNGQPENNYKTTPPVLDS |
| | | DGSFFLYSKLTVDKSRWQQGNVFSCSVMHGALHNHYTQKSLSLSPGK |
| 25 | Mouse Ig gamma-2A chain C | AKTTAPSVYPLAPVCGDTTGSSVTLGCLVKGYFPEPVTLTWNSGSLSSGVHTFPAVLQ |
| | region, A allele | SDLYTLSSSVTVTSSTWPSQSITCNVAHPASSTKVDKKIEPRGPTIKPCPPCKCPAPNL |
| | (UniProt: P01863-1, v1) | LGGPSVFIFPPKIKDVLMISLSPIVTCVVVDVSEDDPDVQISWFVNNVEVHTAQTQTHRE |
| | | DYNSTLRVVSALPIQHQDWMSGKEFKCKVNNKDLPAPIERTISKPKGSVRAPQVYVLP |
| | | PPEEEMTKKQVTLTCMVTDFMPEDIYVEWTNNGKTELNYKNTEPVLDSDGSYFMYSK |
| | | LRVEKKNWVERNSYSCSVVHEGLHNHHTTKSFSRTPGK |
| 26 | G4S linker | GGGGS |
| 27 | (G4S)3 linker | GGGGSGGGGSGGGGS |
| 28 | Human IgG1 constant region | ASTKGPSVFPLAPSSKSTSGGTAALGCLVKDYFPEPVTVSWNSGALTSGVHTFPAVLQ |
| | (G1m3 allotype) | SSGLYSLSSVVTVPSSSLGTQTYICNVNHKPSNTKVDKRVEPKSCDKTHTCPPCPAPE |
| | | LLGGPSVFLFPPKPKDILMISRTPEVTCVVVDVSHEDPEVKFNWYVDGVEVHNAKTKP |
| | | REEQYNSTYRVVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 29 | CH1 IgG1 (G1m3) | ASTKGPSVFPLAPSSKSTSGGTAALGCLVKDYFPEPVTVSWNSGALTSGVHTFPAVLQ |
| | | SSGLYSLSSVVTVPSSSLGTQTYICNVNHKPSNTKVDKRV |
| 30 | CH3 IgG1 (G1m3) | GQPREPQVYTLPPSREEMTKNQVSLTCLVKGFYPSDIAVEWESNGQPENNYKTTPPV |
| | | LDSDGSFFLYSKLTVDKSRVVQQGNVFSCSVMHEALHNHYTQKSLSLSPGK |
| 31 | CH2-CH3 (G1m3) | PCPAPELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKGQP |
| | | REPQVYTLPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |
| 32 | CH3_EKEG (G1m3) | GQPRKPQVYTLPPSREEMTKNQVSLTCLVKGFYPSDIAVEWESNGQPENNYKTTPPV |
| | | LDSDGSFFLYSKLTVDKSRWQQGNVFSCSVMHGALHNHYTQKSLSLSPGK |
| 33 | CH2-CH3_GASD (G1m3) | PCPAPELLAGPDVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKGQP |
| | | REPQVYTLPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |
| 34 | CH2-CH3_GASDIE (G1m3) | PCPAPELLAGPDVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPEEKTISKAKGQP |
| | | REPQVYTLPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |
| 35 | CH2-CH3_GASDALIE | PCPAPELLAGPDVFLFPPKPKDTLMISRTPEVTCVVVDVSHEDPEVKFNWYVDGVEVH |
| | (G1m3) | NAKTKPREEQYNSTYRVVSVLTVLHQDWLNGKEYKCKVSNKALPLPEEKTISKAKGQP |
| | | REPQVYTLPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |
| 36 | CH2-CH3_LCKC (G1m3) | PCPAPELLGGPSVFCFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPIECTISKAKGQ |
| | | PREPQVYTLPPSREEMTKNQVSLTCLVKGFYPSDIAVEWESNGQPENNYKTTPPVLD |
| | | SDGSFFLYSKLTVDKSRWQQGNVFSCSVMHEALHNHYTQKSLSLSPGK |
| 37 | CH2-CH3_GASDLCKC | PCPAPELLAGPDVFCFPPKPKDTLMISRTPEVTCVVVDVSHEDPEVKFNWYVDGVEVH |
| | (G1m3) | NAKTKPREEQYNSTYRVVSVLTVLHQDWLNGKEYKCKVSNKALPAPIECTISKAKGQP |
| | | REPQVYTLPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |
| 38 | CH2-CH3_GASDIELCKC | PCPAPELLAGPDVFCFPPKPKDTLMISRTPEVTCVVVDVSHEDPEVKFNWYVDGVEVH |
| | (G1m3) | NAKTKPREEQYNSTYRVVSVLTVLHQDWLNGKEYKCKVSNKALPAPEECTISKAKGQ |
| | | PREPQVYTLPPSREEMTKNQVSLTCLVKGFYPSDIAVEWESNGQPENNYKTTPPVLD |
| | | SDGSFFLYSKLTVDKSRWQQGNVFSCSVMHEALHNHYTQKSLSLSPGK |
| 39 | CH2-CH3_GASDALIELCKC | PCPAPELLAGPDVFCFPPKPKDTLMISRTPEVTCVVVDVSHEDPEVKFNWYVDGVEVH |
| | (G1m3) | NAKTKPREEQYNSTYRVVSVLTVLHQDWLNGKEYKCKVSNKALPLPEECTISKAKGQP |
| | | REPQVYTLPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |
| 40 | CH2-CH3_EKEG (G1m3) | PCPAPELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKGQP |
| | | RKPQVYTLPPSREEMTKNQVSLTCLVKGFYPSDIAVEWESNGQPENNYKTTPPVLDS |
| | | DGSFFLYSKLTVDKSRWQQGNVFSCSVMHGALHNHYTQKSLSLSPGK |
| 41 | CH2-CH3_LCKCEKEG | PCPAPELLGGPSVFCFPPKPKDTLMISRTPEVTCVVVDVSHEDPEVKFNWYVDGVEV |
| | (G1m3) | HNAKTKPREEQYNSTYRVVSVLTVLHQDWLNGKEYKCKVSNKALPAPIECTISKAKGQ |
| | | PRKPQVYTLPPSREEMTKNQVSLTCLVKGFYPSDIAVEWESNGQPENNYKTTPPVLD |
| | | SDGSFFLYSKLTVDKSRWQQGNVFSCSVMHGALHNHYTQKSLSLSPGK |

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Where a nucleic acid sequence is disclosed herein, the reverse complement thereof is also expressly contemplated.

Methods described herein may preferably performed in vitro. The term “in vitro” is intended to encompass procedures performed with cells in culture whereas the term “in vivo” is intended to encompass procedures with/on intact multi-cellular organisms.

### BRIEF DESCRIPTION OF THE FIGURES

Embodiments and experiments illustrating the principles of the invention will now be discussed with reference to the accompanying figures.

FIGS. 1A to 1E. Graphs showing the first derivative of the raw data obtained by Differential Scanning Fluorimetry analysis of thermostability of antigen-binding molecules comprising different Fc regions. (1A) shows data for WT Fc and LCKC Fc, (1B) shows data for GASD Fc and GASD_LCKC Fc, (1C) shows data for GASDIE Fc and GASDIE_LCKC Fc, (1D) shows data for GASDALIE Fc and GASDALIE_LCKC Fc, and (1E) shows data for EKEG Fc and EKEG_LCKC Fc.

FIGS. 2A and 2B. Table and bar chart summarising the data shown in FIGS. 1A to 1E. (2A) summarises data for all molecules. (2B) provides a graphical representation in the Tm shift in ° C. relative to WT Fc (WT IgG1) for LCKC Fc (LCKC), GASDIE_LCKC Fc (GASDIE-LCKC) and GASDIE Fc (GASDIE) format antigen-binding molecules.

FIGS. 3A to 3J. Sensorgrams showing the binding of antigen-binding molecules comprising different Fc regions to human FcγRIIIa-158V. (3A) shows data for WT Fc, (3B) shows data for GASD Fc, (3C) shows data for GASDIE Fc, (3D) shows data for LCKC Fc, (3E) shows data for GASD_LCKC Fc, (3F) shows data for GASDIE_LCKC Fc, (3G) shows data for GASDALIE Fc, (3H) shows data for EKEG Fc, (3I) shows data for GASDALIE_LCKC Fc, and (3J) shows data for EKEG_LCKC Fc.

FIG. 4. Table summarising the data shown in FIGS. 3A to 3J.

FIGS. 5A to 5J. Sensorgrams showing the binding of antigen-binding molecules comprising different Fc regions to human Fcγ receptors. (5A) shows data for binding of WT Fc to hFcγRIIIa-158F, (5B) shows data for binding of WT Fc to hFcγRIIIa-158V, (5C) shows data for binding of WT Fc to hFcγRIIa-167H, (5D) shows data for binding of GASDALIE_LCKC Fc to hFcγRIIIa-158F, (5E) shows data for binding of GASDALIE_LCKC Fc to hFcγRIIIa-158V, (5F) shows data for binding of GASDALIE_LCKC Fc to hFcγRIIa-167H, (5G) shows data for binding of WT Fc to hFcγRIIa-167R, (5H) shows data for binding of WT Fc to hFcγRIIb, (5I) shows data for binding of GASDALIE_LCKC Fc to hFcγRIIa-167R, and (5J) shows data for binding of GASDALIE_LCKC Fc to hFcγRIIb.

FIGS. 6A to 6F. Sensorgrams showing the binding of antigen-binding molecules comprising different Fc regions to mouse Fcγ receptors. (6A) shows data for binding of WT Fc to mFcγRIV, (6B) shows data for binding of WT Fc to mFcγRIII, (6C) shows data for binding of WT Fc to mFcγRIIb, (6D) shows data for binding of GASDALIE_LCKC Fc to mFcγRIV, (6E) shows data for binding of GASDALIE_LCKC Fc to mFcγRIII, and (6F) shows data for binding of GASDALIE_LCKC Fc to mFcγRIIb.

FIGS. 7A to 7D. Sensorgrams showing the binding of antigen-binding molecules comprising different Fc regions to human (h) and mouse (m) FcRn receptors. (7A) shows data for binding of WT Fc to hFcRn, (7B) shows data for binding of WT Fc to mFcRn, (7C) shows data for binding of GASDALIE_LCKC Fc to hFcRn, and (7D) shows data for binding of GASDALIE_LCKC Fc to mFcRn.

FIG. 8. Table summarising the data shown in FIGS. 5A to 5J, 6A to 6F and 7A to 7D.

FIGS. 9A and 9B. Graph and bar chart showing ADCC mediated by antigen-binding molecules comprising different Fc regions to target antigen-expressing cells, as determined by LDH release assay. (9A) shows ADCC activity of WT Fc or GASDALIE_LCKC Fc regions to target antigen-expressing cells. EC50 values are shown. (9B) shows relative ADCC activity of WT Fc, GASDALIE_LCKC Fc or N297Q Fc to target antigen-expressing cells.

FIG. 10. Graph showing the results of analysis of tumour volume over time in an A549 cell-line derived mouse model of lung adenocarcinoma. Antigen-binding molecules comprising WT Fc or GASDALIE_LCKC Fc were administered IP, biweekly at 25 mg/kg for a total of 6 weeks. A control treatment group received an equal volume of PBS (vehicle).

### EXAMPLES

### Example 1

### Preparation of Antigen-Binding Molecules Comprising Engineered Fc Regions

The inventors prepared antigen-binding molecules comprising heavy chains including amino acid substitutions to positions in the CH2 and/or CH3 regions, to investigate the consequence of the substitutions on Fc effector functions.

Antigen-binding molecules were prepared comprising: (i) light chains comprising the light chain variable region (VL) of an antibody specific for HER3, and the constant region light chain (Cκ), and (ii) heavy chains comprising the heavy chain variable region (VH) of the antibody specific for HER3, and human immunoglobulin G 1 (G1m3 allotype) heavy chain constant region 1 (CH1), hinge region, heavy chain constant region 2 (CH2) and heavy chain constant region 3 (CH3).

The CH2 and CH3 regions were either unsubstituted, or were provided with combinations of substitutions, as follows:

| | Substitutions relative | Amino acid | Amino acid | Amino acid |
|---|---|---|---|---|
| Fc region | to human IGHG1 | sequence of | sequence of | sequence of |
| designation | (G1m3 allotype) | CH2 region | CH3 region | CH2-CH3 region |
| Wildtype (WT) | — | SEQ ID NO: 4 | SEQ ID NO: 30 | SEQ ID NO: 31 |
| GASD | G236A, S293D | SEQ ID NO: 8 | SEQ ID NO: 30 | SEQ ID NO: 33 |
| GASDIE | G236A, S293D, | SEQ ID NO: 9 | SEQ ID NO: 30 | SEQ ID NO: 34 |
| | I332E | | | |
| GASDALIE | G236A, S293D, | SEQ ID NO: 10 | SEQ ID NO: 30 | SEQ ID NO: 35 |
| | A330L, I332E | | | |
| EKEG | E345K, E430G | SEQ ID NO: 4 | SEQ ID NO: 32 | SEQ ID NO: 40 |
| LCKC | L242C, K334C | SEQ ID NO: 11 | SEQ ID NO: 30 | SEQ ID NO: 36 |
| GASD_LCKC | G236A, S293D, | SEQ ID NO: 12 | SEQ ID NO: 30 | SEQ ID NO: 37 |
| | L242C, K334C | | | |
| GASDIE_LCKC | G236A, S293D, | SEQ ID NO: 13 | SEQ ID NO: 30 | SEQ ID NO: 38 |
| | I332E, L242C, | | | |
| | K334C | | | |
| GASDALIE_LCKC | G236A, S293D, | SEQ ID NO: 14 | SEQ ID NO: 30 | SEQ ID NO: 39 |
| | A330L, I332E, | | | |
| | L242C, K334C | | | |
| EKEG_LCKC | E345K, E430G, | SEQ ID NO: 11 | SEQ ID NO: 32 | SEQ ID NO: 41 |
| | L242C, K334C | | | |

Antigen-binding molecules were expressed using either 1) Expi293 Transient Expression System Kit (Life Technologies, USA), or 2) HEK293-6E Transient Expression System (CNRC-NRC, Canada) following the manufacturer's instructions.

1) Expi293 Transient Expression System:

Cell Line Maintenance:

HEK293F cells (Expi293F) were obtained from Life Technologies, Inc (USA). Cells were cultured in serum-free, protein-free, chemically defined medium (Expi293 Expression Medium, Thermo Fisher, USA), supplemented with 50 IU/ml penicillin and 50 μg/ml streptomycine (Gibco, USA) at 37° C., in 8% CO₂ and 80% humidified incubators with shaking platform.

Transfection:

Expi293F cells were transfected with expression plasmids encoding the heavy and light chains using ExpiFectamine 293 Reagent kit (Gibco, USA) according to its manufacturer's protocol. Briefly, cells at maintenance were subjected to a media exchange to remove antibiotics by spinning down the culture, cell pellets were re-suspended in fresh media without antibiotics at 1 day before transfection. On the day of transfection, 2.5×10⁶/ml of viable cells were seeded in shaker flasks for each transfection. DNA-ExpiFectamine complexes were formed in serum-reduced medium, Opti-MEM (Gibco, USA), for 25 min at room temperature before being added to the cells. Enhancers were added to the transfected cells at 16-18 h post transfection. An equal amount of media was topped up to the transfectants at day 4 post-transfection to prevent cell aggregation. Transfectants were harvested at day 7 by centrifugation at 4000×g for 15 min, and filtered through 0.22 μm sterile filter units.

2) HEK6293-6E Transient Expression System:

Cell Line Maintenance:

HEK293-6E cells were obtained from National Research Council Canada. Cells were cultured in serum-free, rotein-free, chemically defined Freestyle F17 Medium (Invitrogen, USA), supplemented with 0.1% Kolliphor-P188 and 4 mM L-Glutamine (Gibco, USA) and 25 μg/ml G-418 at 37° C., in 5% CO₂ and 80% humidified incubators with shaking platform.

Transfection:

HEK293-6E cells were transfected with expression plasmids encoding the heavy and light chains using PElpro™ (Polyplus, USA) according to its manufacturer's protocol. Briefly, cells at maintenance were subjected to a media exchange to remove antibiotics by centrifugation, cell pellets were re-suspended with fresh media without antibiotics at 1 day before transfection. On the day of transfection, 1.5-2×10⁶ cells/ml of viable cells were seeded in shaker flasks for each transfection. DNA and PElpro™ were mixed to a ratio of 1:1 and the complexes were allowed to form in F17 medium for 5 min at RT before adding to the cells. 0.5% (w/v) of Tryptone N1 was fed to transfectants at 24-48 h post transfection. Transfectants were harvested at day 6-7 by centrifugation at 4000×g for 15 min and the supernatant was filtered through 0.22 μm sterile filter units.

Affinity purification, buffer exchange and storage:

Antigen-binding molecules secreted by the transfected cells into the culture supernatant were purified using liquid chromatography system AKTA Start (GE Healthcare, UK). Specifically, supernatants were loaded onto HiTrap Protein G column (GE Healthcare, UK) at a binding rate of 5 ml/min, followed by washing the column with 10 column volumes of washing buffer (20 mM sodium phosphate, pH 7.0). Bound mAbs were eluted with elution buffer (0.1 M glycine, pH 2.7) and the eluents were fractionated to collection tubes which contain appropriate amount of neutralization buffer (1 M Tris, pH 9). Neutralised elution buffer containing purified mAb were exchanged into PBS using 30K MWCO protein concentrators (Thermo Fisher, USA) or 3.5K MWCO dialysis cassettes (Thermo Fisher, USA). Monoclonal antibodies were sterilized by passing through 0.22 μm filter, aliquoted and snap-frozen in -−0° C. for storage.

### Example 2

### Analysis of Thermostability of Antigen-Binding Molecules Comprising Engineered Fc regions by Differential Scanning Fluorimetry

Thermostability of the antigen-binding molecules prepared as described in Example 1 was evaluated by Differential Scanning Fluorimetry.

Briefly, triplicate reaction mixes of antibodies at 0.2 mg/mL and SYPRO Orange dye (ThermoFisher) were prepared in 25 μL of PBS, transferred to wells of MicroAmp Optical 96-Well Reaction Plates (ThermoFisher), and sealed with MicroAmp Optical Adhesive Film (ThermoFisher). Melting curves were run in a 7500 fast Real-Time PCR system (Applied Biosystems) selecting TAMRA as reporter and ROX as passive reference. The thermal profile included an initial step of 2 min at 25° C. and a final step of 2 min at 99° C., with a ramp rate of 1.2%. The first derivative of the raw data was plotted as a function of temperature to obtain the derivative melting curves.

Melting temperatures (Tm) for unpairing of the heavy chains were determined from the peaks of the derivative curves.

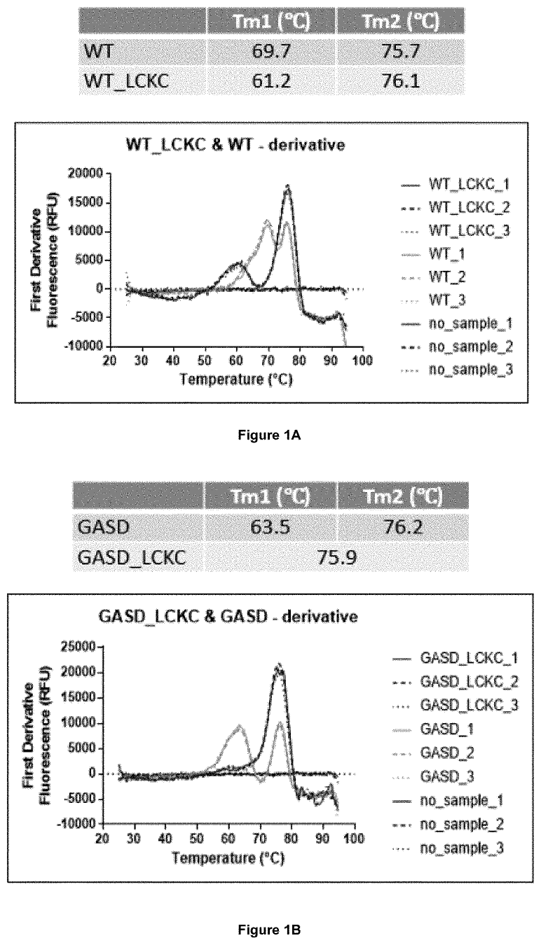

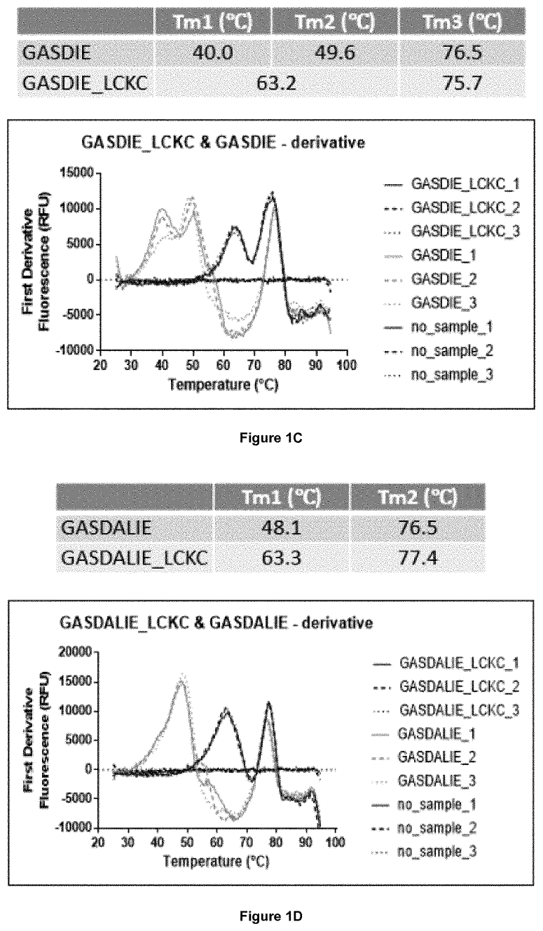

The results are shown in FIGS. 1A to 1E.

Tm values for unpairing of the light chains from the heavy chains in the Fab regions of the antigen-binding molecules were also determined.

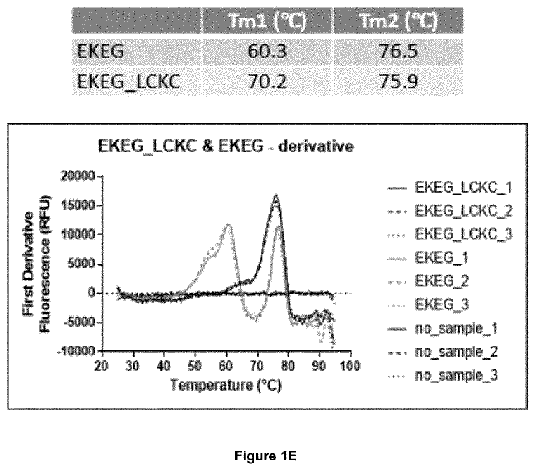

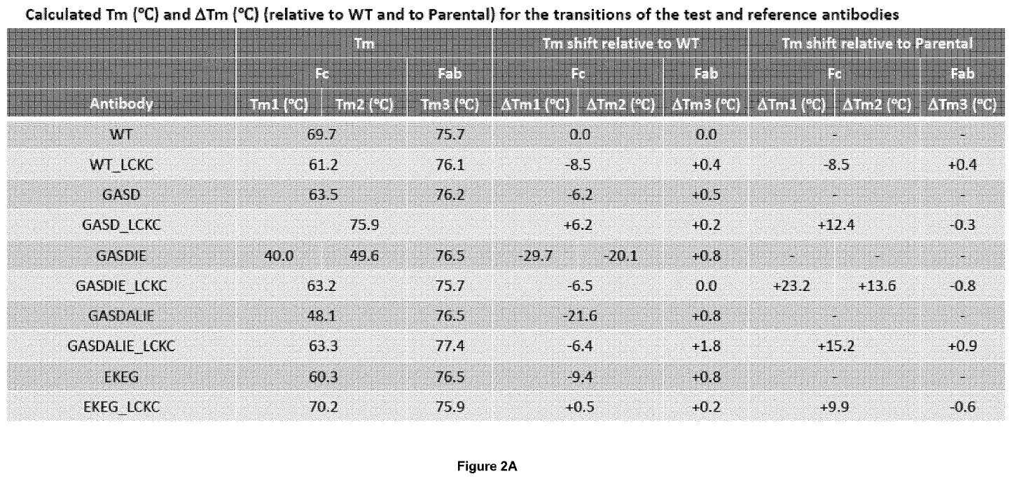

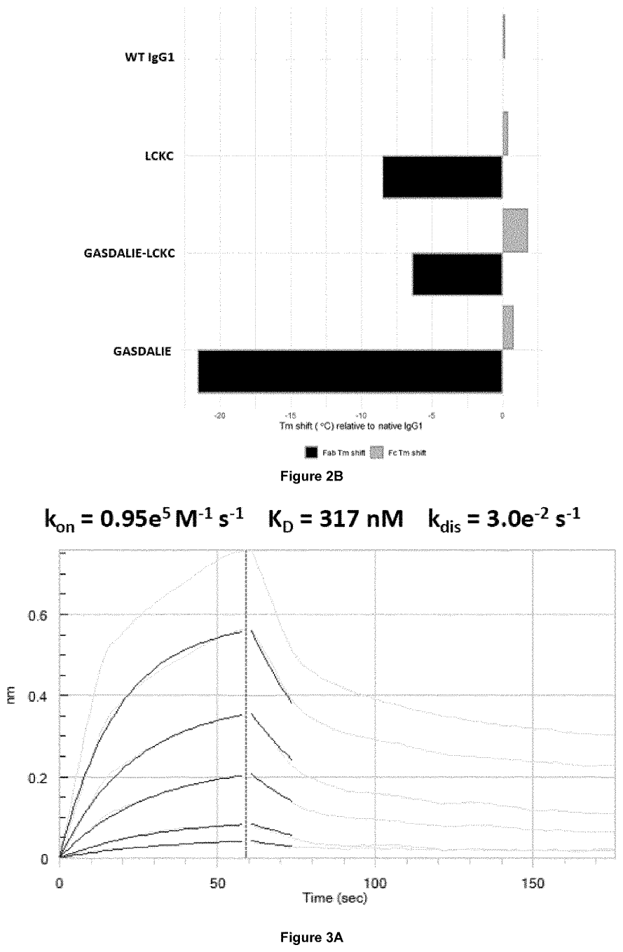

The results of the Differential Scanning Fluorimetry experiments are summarised in the table shown in FIG. 2A.

The introduction of the LCKC substitutions stabilised the Fc region of all the engineered variants tested (i.e. GASD, GASDIE, GASDALIE, EKEG), but destabilised the WT Fc region. LCKC increased the Tm of the engineered Fc variants between 9.9° C. and 23.2° C. LCKC decreased the Tm of the WT Fc by 8.5° C.

The rank order of the thermostability of the engineered Fc variants lacking the LCKC substitutions was as follows: WT (69.7° C.)>GASD (63.6° C.)>EKEG (60.3° C.)>GASDALIE (48.1° C.)≈GASDIE (40.0° C.).

The rank order of the thermostability of the engineered Fc variants comprising the LCKC substitutions was as follows: GASD_LCKC (75.9° C.)>EKEG_LCKC (70.2° C.)>GASDALIE_LCKC (63.3≈GASDIE_LCKC (63.2° C.)>WT_LCKC (61.2° C.).

The change in thermostability of the engineered Fc variants with the LCKC substitutions relative to the Fc WT was as follows: GASD_LCKC (+6.2° C.)>EKEG_LCKC (+0.5° C.)>GASDALIE_LCKC (−6.4° C.)≥GASDIE_LCKC (−6.5° C.).

Introduction of the LCKC substitutions did not affect significantly the thermostability of the Fab region.

### Example 3

### Analysis of Affinity of Antigen-Binding Molecules Comprising Engineered Fc Regions for Human Fc Receptor FcγRIIIa-158V

The antigen-binding molecules prepared as described in Example 1 were evaluated for binding to human Fc receptor FcγRIIIa comprising the polymorphism 158V, by Biolayer Interferometry (BLI) using a Pall ForteBio Octet Red384 system.

Anti-Penta-HIS (HIS1K) biosensors were purchased from Forte Bio (18-5120), and were incubated for 60 sec in PBS buffer (pH 7.2) to obtain the first baseline, and were subsequently loaded for 120 sec with histidine-tagged human FcγRIIIa-158V in PBS pH 7.2. After loading, biosensors were incubated for 60 sec in PBS buffer (pH 7.2) to obtain the second baseline, followed by incubation for 60 sec with a dilution series of the test antigen-binding molecules at concentrations ranging from 15.6 nM to 500 nM in PBS pH 7.2, to obtain association curves. Finally, the biosensors were incubated for 120 sec in PBS pH 7.2 to obtain dissociation curves.

Kinetic and affinity constants were calculated by global fitting of the association and dissociation data to a 1:1 binding model.

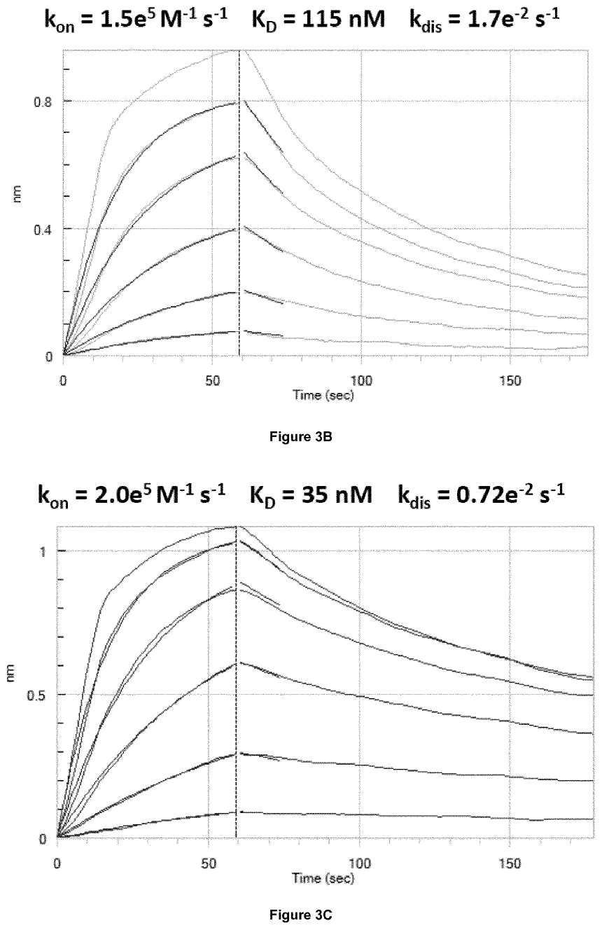

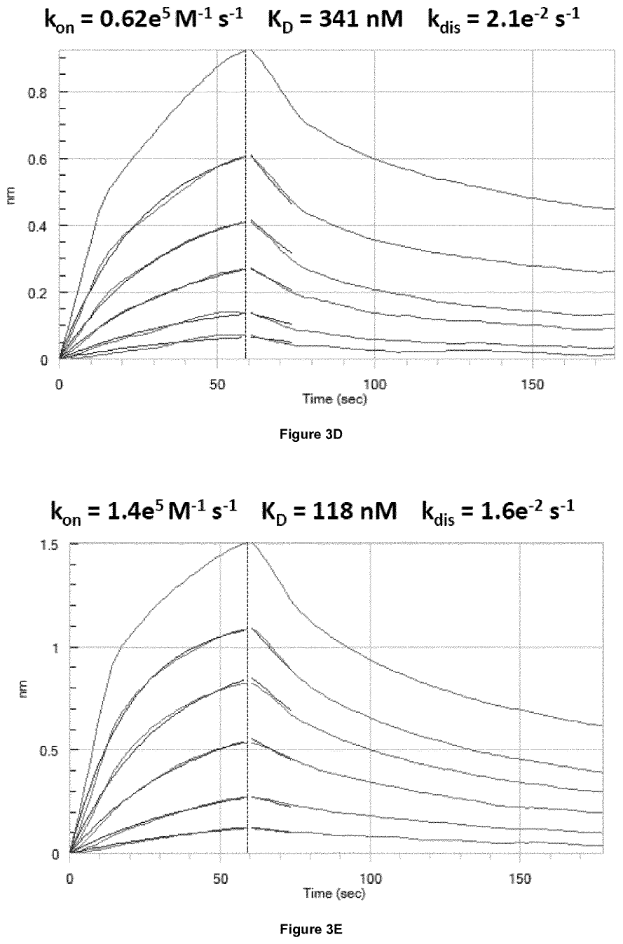

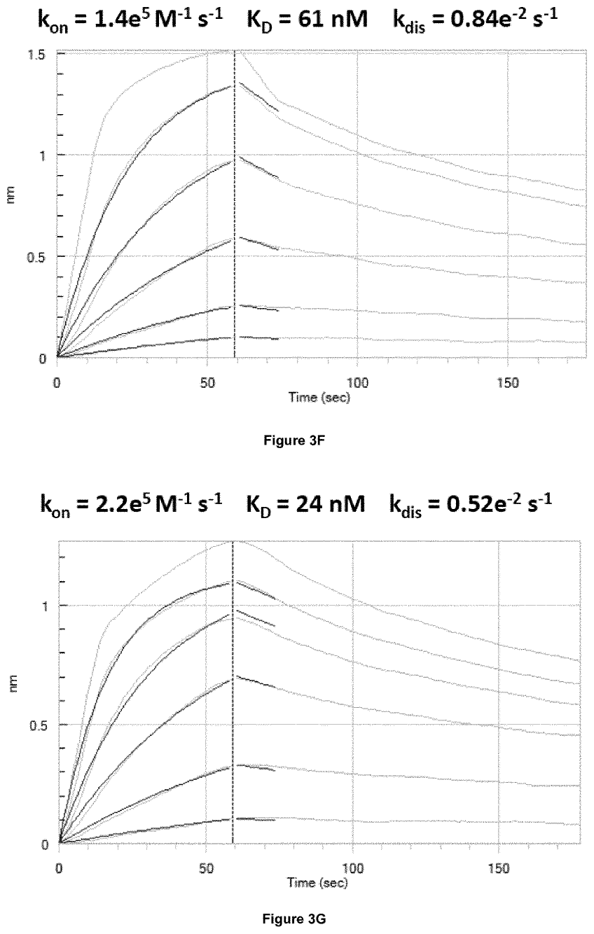

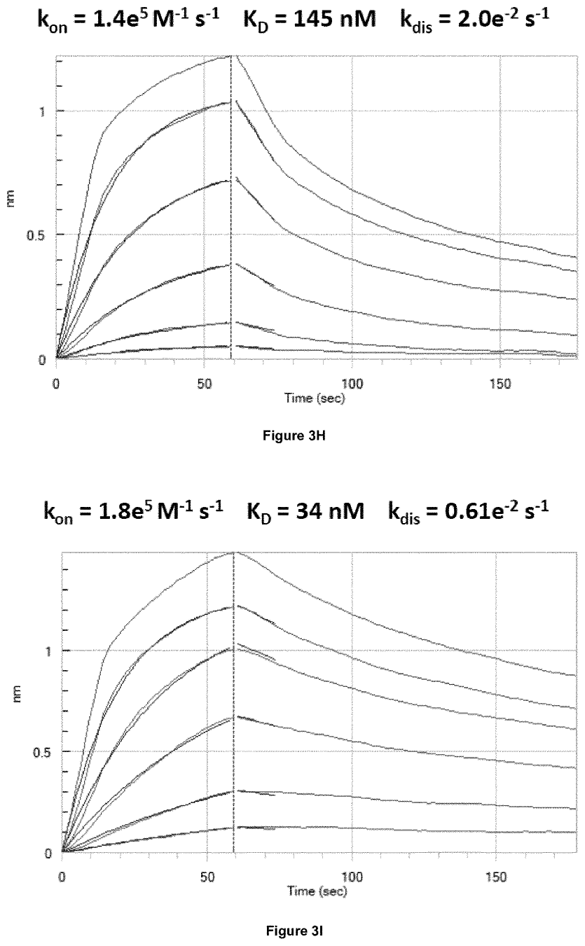

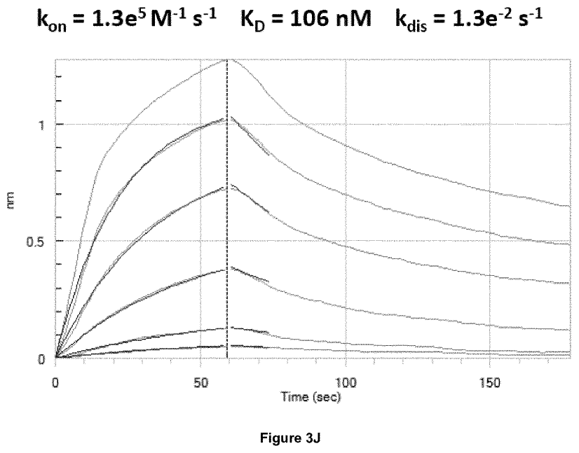

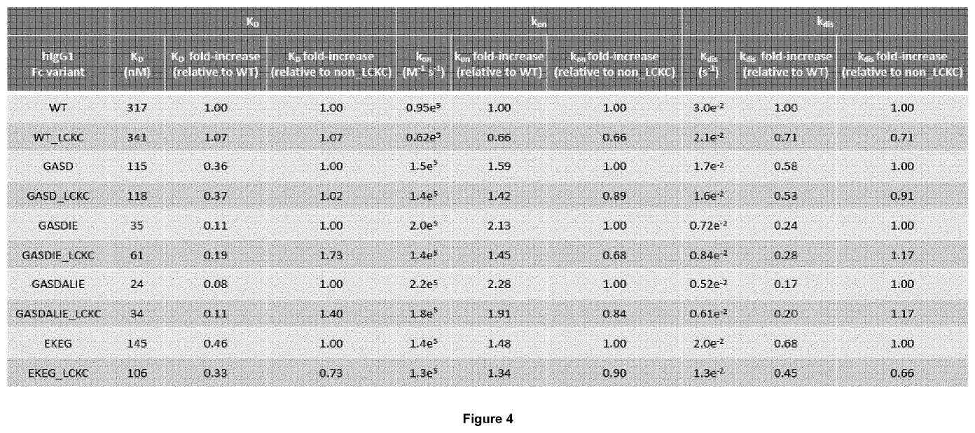

The results are shown in FIGS. 3A to 3J, and are summarized in the table shown in FIG. 4.

GASD, GASDIE, GASDALIE and EKEG Fc variants displayed increased binding affinity to human FcγRIIIa-158V as compared to WT Fc.

The rank order of affinities was as follows: GASDALIE≈GASDIE>GASD≈EKEG>WT.

The observed increase in binding affinity for the engineered Fc variants appeared to mainly be as a consequence of a decrease in the dissociation kinetics.

Introduction of LCKC substitutions did not significantly modify the binding affinity of the WT Fc or GASD, GASDIE, GASDALIE and EKEG Fc variants to human FcγRIIIa-158V.

The rank order of affinities was the same as for the molecules lacking the LCKC substitutions (i.e. GASDALIE_LCKC≈GASDIE_LCKC>GASD_LCKC≈EKEG_LCKC>WT_LCKC).

### Example 4

### Analysis of Affinity of Antigen-Binding Molecules Comprising Engineered Fc Regions for Human and Mouse Fc Receptors

The antigen-binding molecules WT and GASDALIE_LCKC prepared as described in Example 1 were analysed by Biolayer Interferometry (BLI) using a Pall ForteBio Octet Red384 system, for binding to:
human Fc receptors: hFcγRIIIa-158F, hFcγRIIIa-158V, hFcγRIIa-167H, hFcγRIIa-167R, hFcγRIIb and hFcRn; and
mouse Fc receptors: mFcγRIV (orthologue of hFcγRIIIa), mFcγRIII (orthologue of hFcγRIIa), mFcγRIIb (orthologue of hFcγRIIb) and mFcRn.

Anti-Penta-HIS (HIS1K) biosensors were incubated for 60 sec in PBS buffer to obtain the first baseline, and were subsequently loaded for 120 sec with histidine-tagged Fc receptors in PBS. After loading, biosensors were incubated for 60 sec in PBS buffer (pH 7.2 for Fcγ receptors and pH 5.8 for FcRn) to obtain the second baseline, followed by incubation for 60 sec with a dilution series of the test antigen-binding molecules in PBS (pH 7.2 for Fcγ receptors and pH 5.8 for FcRn), at concentrations ranging from 125 nM to 4000 nM (for experiments investigating binding to Fcγ receptors), or 75 nM to 1000 nM (for experiments investigating binding to FcRn receptors), to obtain association curves. Finally, the biosensors were incubated for 120 sec in PBS (pH 7.2 for Fcγ receptors and pH 5.8 for FcRn) to obtain dissociation curves.

PBS pH 7.2 was used for experiments investigating binding to Fcγ receptors, and PBS pH 5.8 was used for experiments investigating binding to FcRn receptors. Kinetic and affinity constants were calculated by global fitting of the association and dissociation data to a 1:1 binding model.

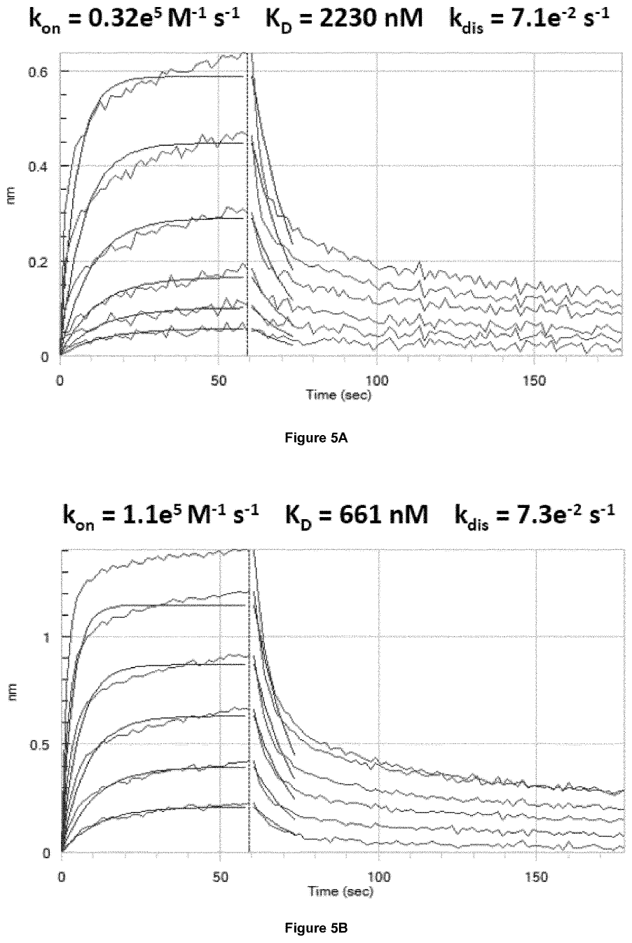

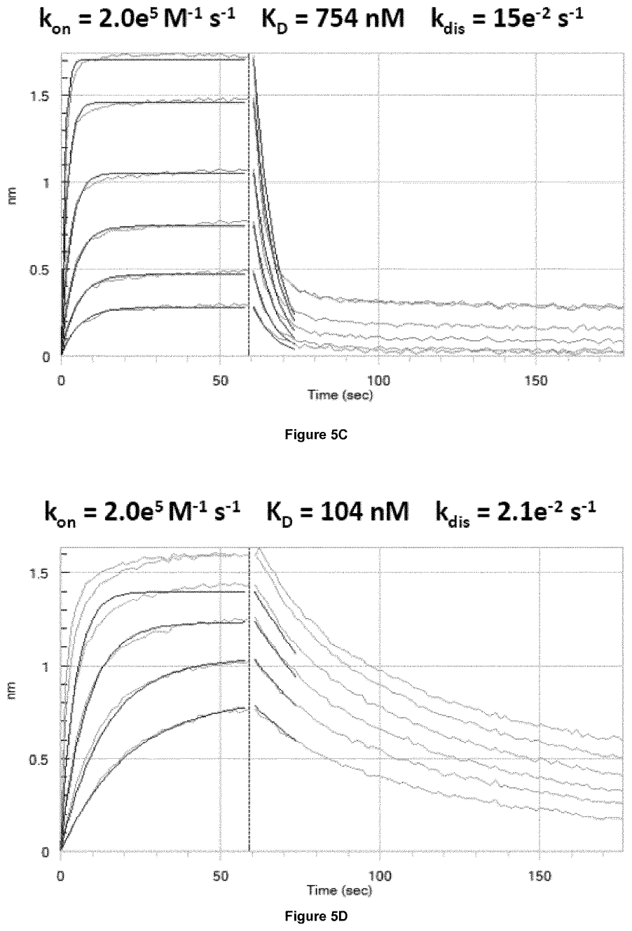

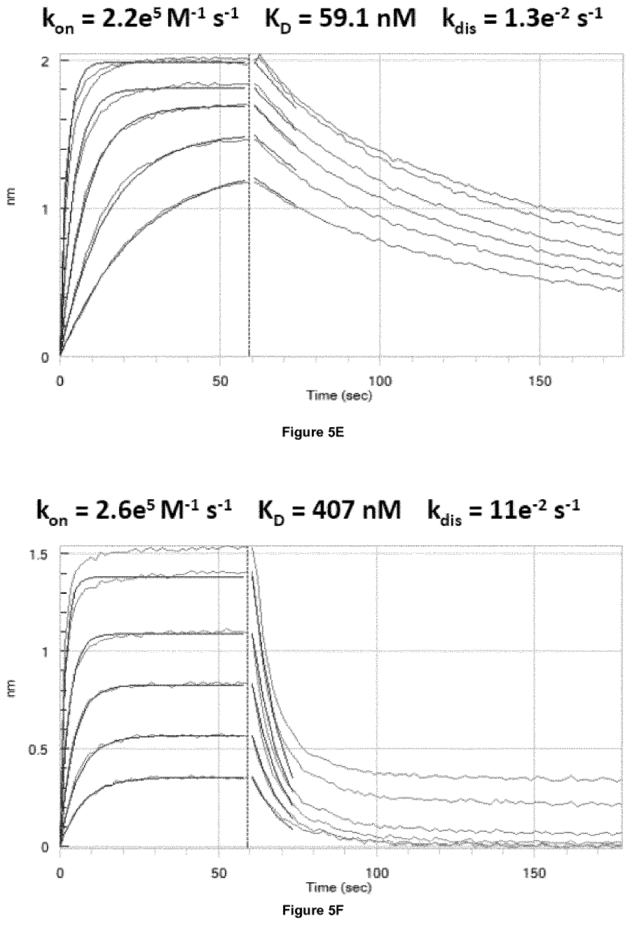

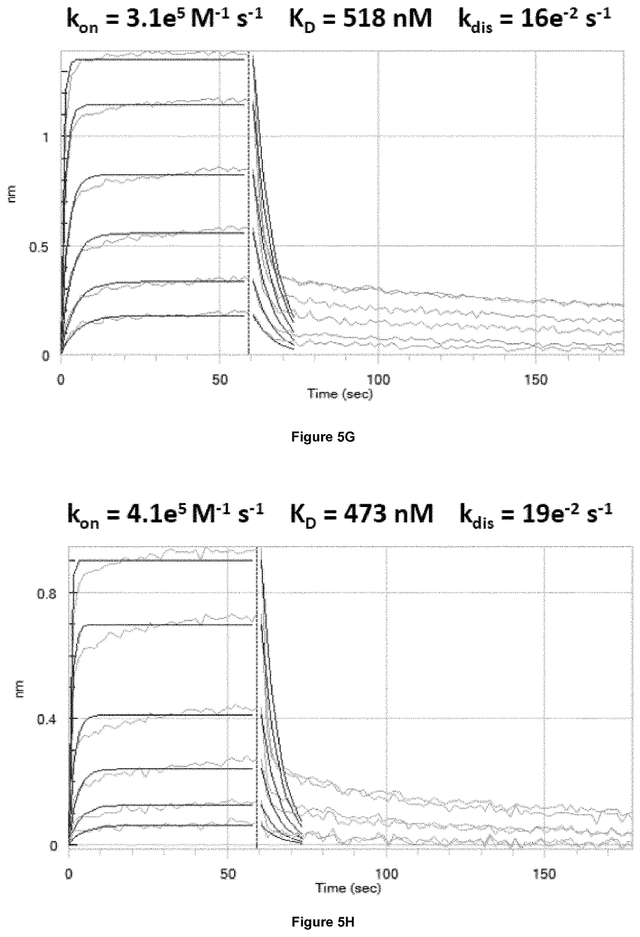

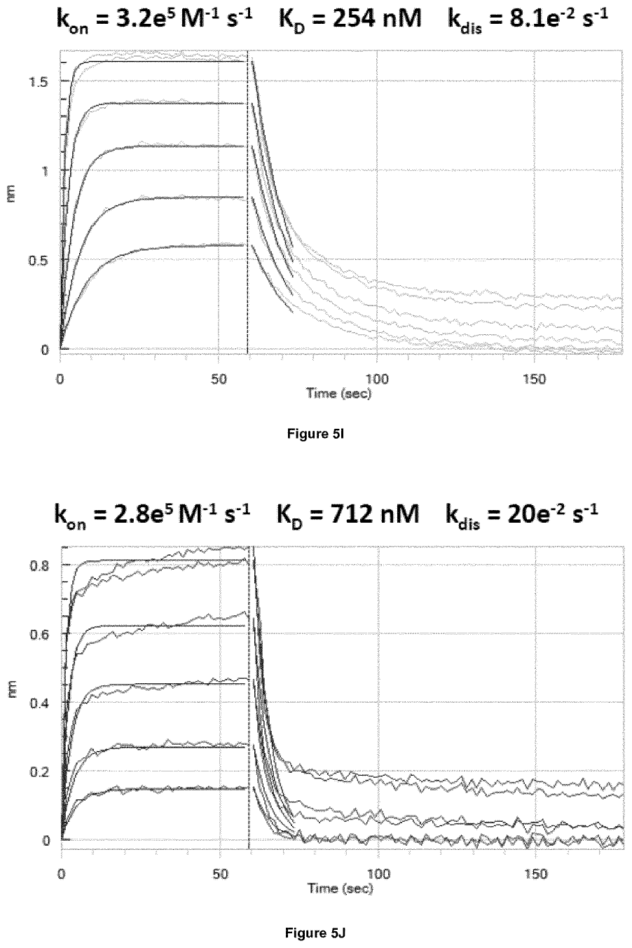

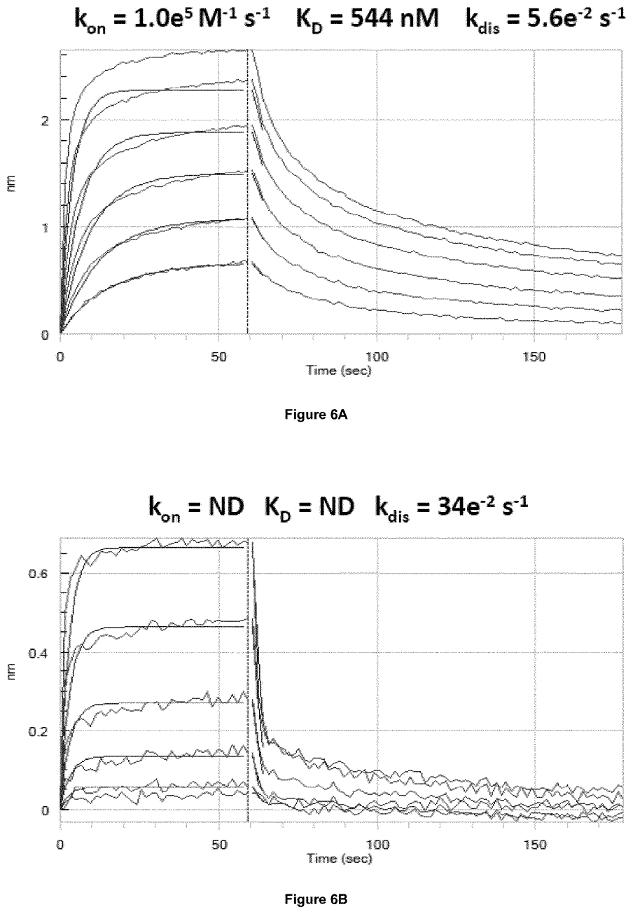

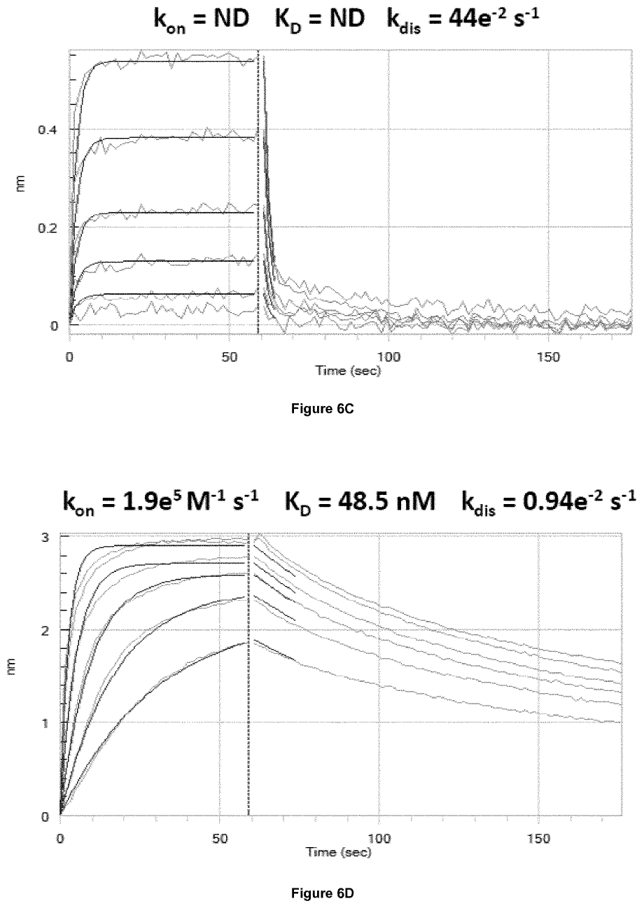

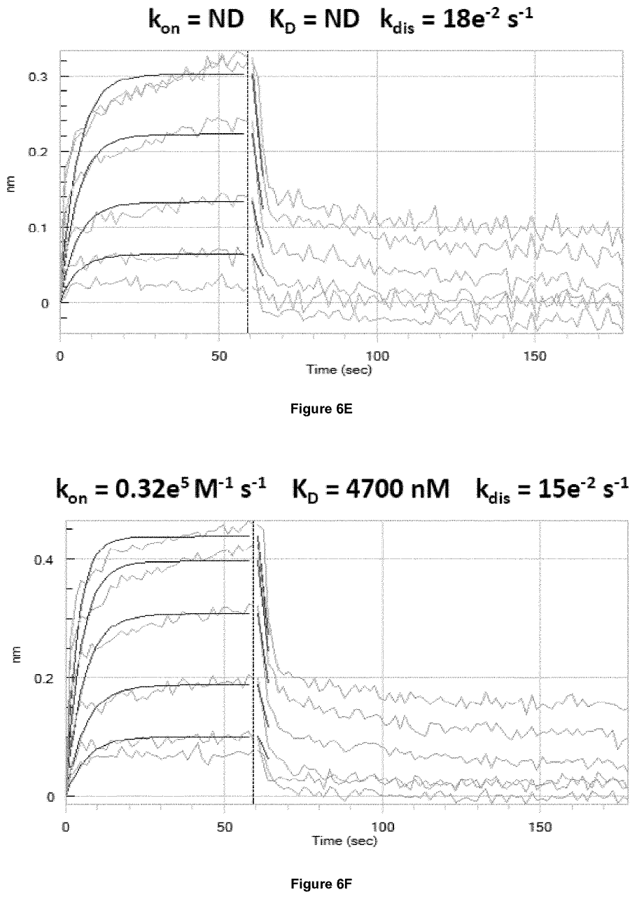

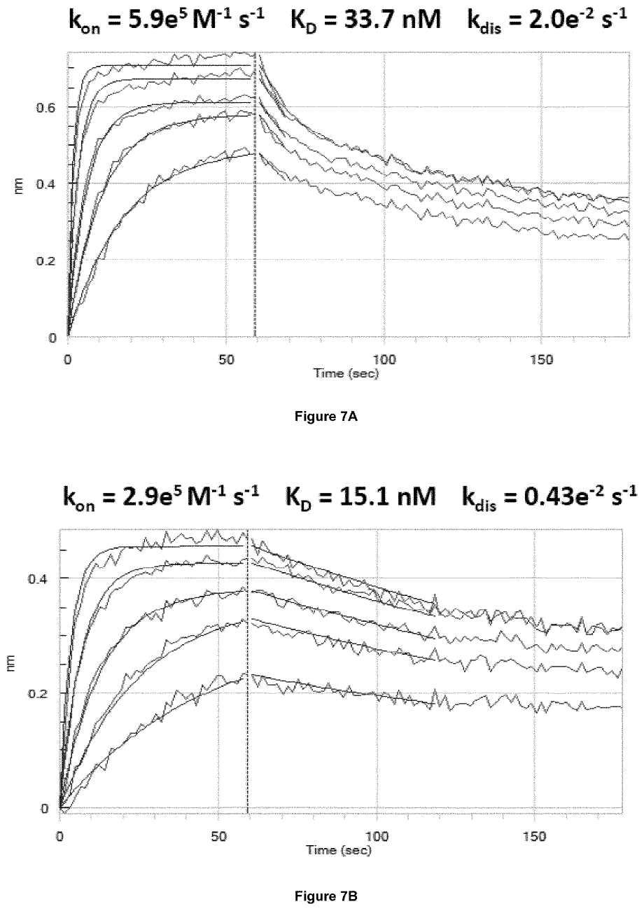

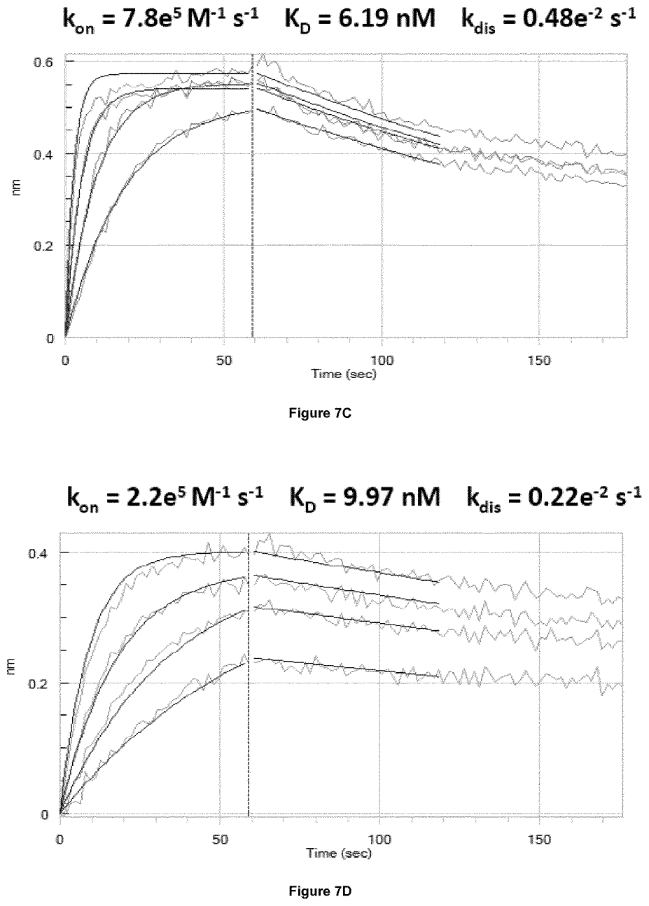

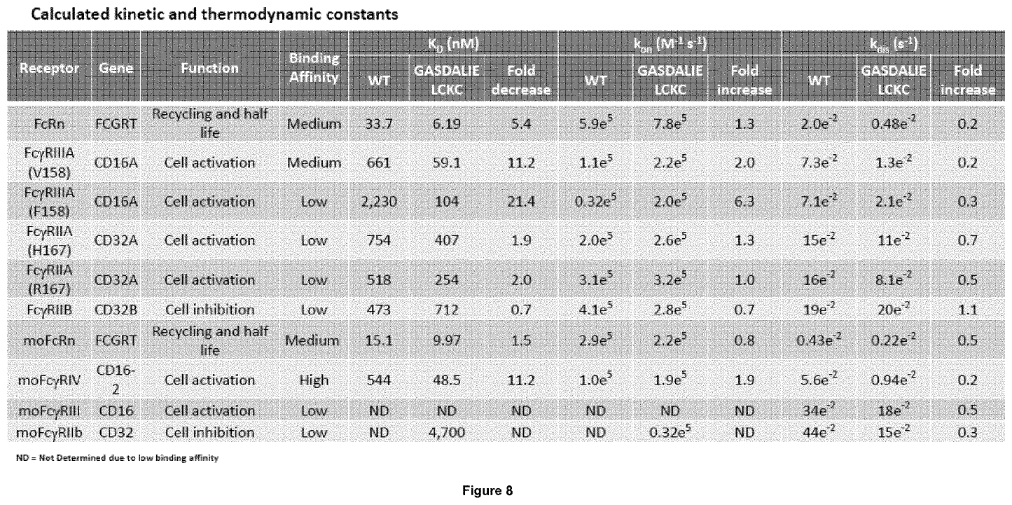

The results are shown in FIGS. 5A to 5J, FIGS. 6A to 6F, 7A to 7D and are summarized in the table shown in FIG. 8.

The GASDALIE_LCKC variant Fc displayed increased affinity to the activatory Fcγ receptors and FcRn receptors relative to WT Fc: hFcγRIIIa-158F, hFcγRIIIa-158V, hFcγRIIa-167H and hFcγRIIa-167R, hFcRn, mFcγRIV and mFcRn.

The GASDALIE_LCKC variant Fc displayed decreased affinity to the inhibitory Fcγ receptor hFcγRIIb relative to WT Fc.

The GASDALIE_LCKC variant Fc did not differ significantly relative to WT Fc in its affinity for binding to mFcγRIII and mFcγRIIb. Overall, the GASDALIE_LCKC variant Fc displayed increased affinity for human and mouse activatory Fcγ receptors and FcRn, and increased selectivity for human activatory Fcγ receptors as compared to human inhibitory Fcγ receptors.

### Example 5

### Analysis of Antibody-Dependent Cell-Mediated Cytotoxicity (ADCC) of Antigen-Binding Molecules Comprising Engineered Fc Regions

The antigen-binding molecules WT and GASDALIE_LCKC prepared as described in Example 1 were analysed for their ability to cause ADCC to cells expressing the target antigen (HER3), in an in vitro assay.

HEK 293 T cells stably transfected with constructs encoding human HER3 were used as target cells in the assay (expression of HER3 at the cell surface was confirmed by analysis by flow cytometry using a HER3-specific antibody).

Briefly, target cells were plated in wells of 96 well U-bottom plates at a density of 20,000 cells/well. Cells were incubated with WT or GASDALIE_LCKC antigen-binding molecules in a dilution series with final concentrations ranging from 50,000 ng/ml to 0.18 ng/ml (50,000 ng/ml, 8,333 ng/ml, 1,389 ng/ml, 231 ng/ml, 38.6 ng/ml, 6.4 ng/ml, 1.1 ng/ml and 0.18 ng/ml), or were left untreated. The cells were incubated at 37° C. and at 5% CO₂ for 30 min.

Effector cells (Human Natural Killer Cell Line NoGFPCD16.NK92; 176V) were subsequently added to the wells at a density of 60,000 cells/well (i.e. the effector:target cell ratio was 3:1).

The following control conditions were included: target cell maximal LDH release (contained target cells only), spontaneous release (contained target cells and effectors cells, in the absence of antigen-binding molecules) and background (cell culture media only).

Plates were span down and incubated at 37° C. and at 5% CO2 for 21 hours. LDH Release Assay was performed using the Pierce LDH Cytotoxicity Assay Kit. 10 μl of Lysis Buffer (10×) was added to target cell maximal LDH release control wells, and incubated at 37° C. and 5% CO₂ for 20 min. After incubation, plates were span down, and 50 ml of the supernatant was transferred to clear flat-bottom 96-well plates. Reactions were started by addition of 50 μl of LDH substrate-containing assay mix to the supernatants, and reactions were incubated at 37° C. for 30 min. Reactions were stopped by addition of 50 μl of stop solution, and absorbance at 490 nm and 680 nm was recorded using a BioTek Synergy HT microplate reader.

Absorbance from test samples were corrected to values obtained for background and spontaneous release control conditions, and percent cytotoxicity was calculated relative to target cell maximal LDH release control, and plotted as a function of antibody concentration. EC50 values (ng/ml) were calculated.

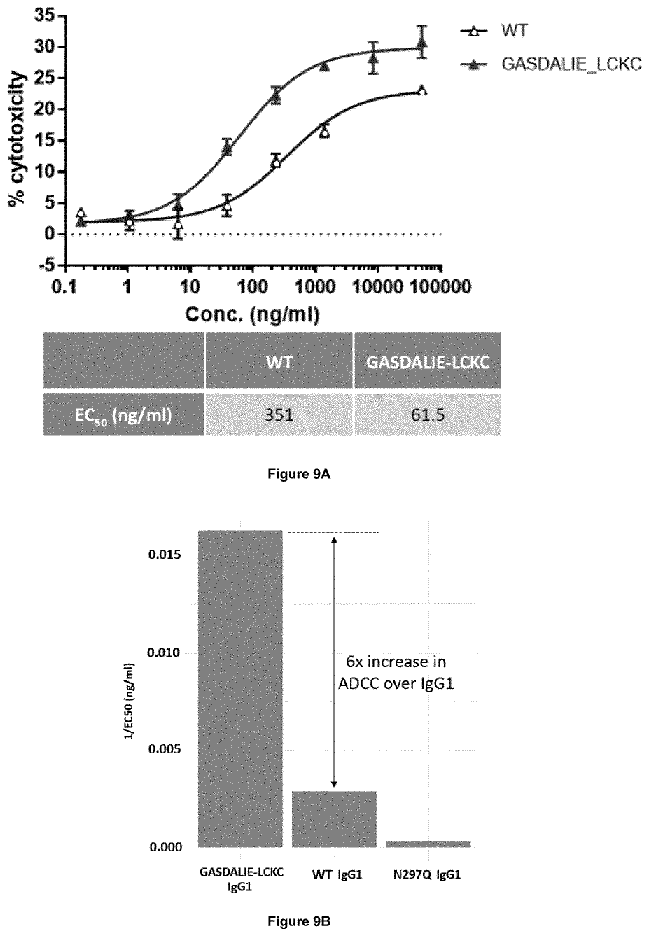

The results are shown in FIGS. 9A and 9B. Antigen-binding molecules comprising both WT Fc and GASDALIE_LCKC Fc elicited concentration-dependent ADCC to cells expressing the target antigen. Antigen-binding molecules comprising GASDALIE_LCKC Fc had an increased maximum cytotoxicity as compared to antigen-binding molecules comprising WT Fc, and were potent having a 6-fold decrease in EC50 relative to WT (FIG. 9B).

### Example 6

### Analysis of Tumor Growth Inhibition In Vivo by Antigen-Binding Molecules Comprising Engineered Fc Regions

Female NCr nude mice approximately 6-8 weeks old were purchased from In Vivos (Singapore). Animals were housed under specific pathogen-free conditions and were treated in compliance with the Institutional Animal Care and Use Committee (IACUC) guidelines.

Ectopic tumors were established by subcutaneous injection of 5×10⁶ A549 cells (HER3-expressing lung cancer cells) into the right flank. Mice were administered biweekly with IP injection of 25 mg/kg of the antigen-binding molecule comprising WT-Fc (n=6), or the antigen-binding molecule comprising the GASDALIE_LCKC substitutions (n=6), for a total of 6 weeks. A control treatment group received an equal volume of PBS (vehicle; n=8).

Tumor volumes were measured 3 times a week using a digital caliper and calculated using the formula [L×W2/2]. Study End point was considered to have been reaches once the tumors of the control arm measured >1.5 cm in length.

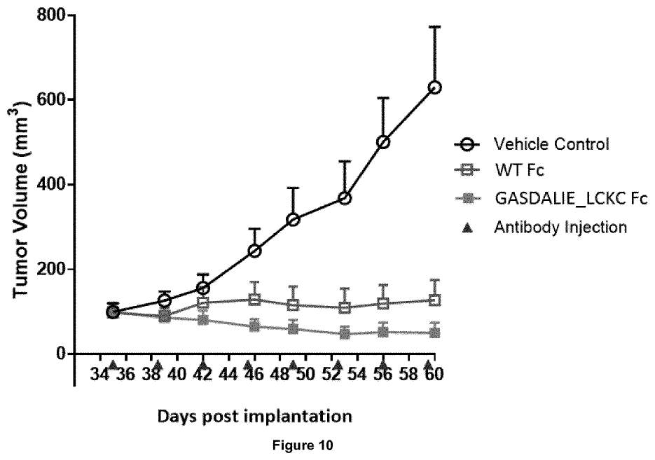

The results are shown in FIG. 10. The antigen-binding molecule comprising the GASDALIE_LCKC substitutions was found to be significantly more potent at inhibiting tumor growth than the antigen-binding molecule comprising WT-Fc.

### Example 7

### Analysis of Possible Sequence Liabilities/Immunogenic Sequences Introduced by GASDALIE LCKC Substitutions

The inventors next investigated whether introducing the GASDALIE_LCKC substitutions into humanized IgG1 antibody trastuzumab influenced properties relevant to antibody production, or use in therapy.

The inventors investigated whether the GASDALIE_LCKC substitutions were predicted to affect N-glycosylation, O-glycosylation, C-mannosylation, Asn deamidation, solubility and immunogenicity by in silico analysis of the amino acid sequences of the constituent polypeptides of trastuzumab, and the trastuzumab variant comprising GASDALIE_LCKC substitutions.

The introduction of GASDALIE_LCKC substitutions into the Fc region of trastuzumab was predicted not to affect N-glycosylation, O-glycosylation, C-mannosylation, Asn deamidation, nor to introduce any immunogenic peptides, and was predicted to result in a 2% increase in solubility.

### Example 8

### Conclusion

The antigen-binding molecules comprising GASDALIE_LCKC Fc were demonstrated to be provided with the following combination of advantageous properties relative to WT Fc:
(i) increased affinity for activatory Fcγ receptors(>12 times increase in binding to human FcγRIIIa activatory receptors, and similar binding to murine FcγRIV activatory receptors);
(ii) increased affinity for the neonatal Fc receptor FcRn;
(iii) decreased affinity for inhibitory Fcγ receptors;
(iv) increased selectivity for activatory Fcγ receptors vs. inhibitory Fcγ receptors
(v) similar thermostability
(vi) increased ADCC activity in vitro (6 times increase)
(vii) improved tumor growth inhibition in vivo;
(viii) no additional sequence liabilities/immunogenicity.

1. An antigen-binding molecule, optionally isolated, comprising an Fc region, the Fc region comprising a polypeptide having: (i) C at the position corresponding to position 242, and C at the position corresponding to position 334, and (ii) one or more of: A at the position corresponding to position 236, D at the position corresponding to position 239, E at the position corresponding to position 332, L at the position corresponding to position 330, K at the position corresponding to position 345, and G at the position corresponding to position 430.

2. The antigen binding molecule according to claim 1, wherein the Fc region comprises a polypeptide having: (i) C at the position corresponding to position 242, and C at the position corresponding to position 334, and (ii) A at the position corresponding to position 236, D at the position corresponding to position 239, E at the position corresponding to position 332, and L at the position corresponding to position 330;
or A at the position corresponding to position 236, D at the position corresponding to position 239, and E at the position corresponding to position 332; or A at the position corresponding to position 236, and D at the position corresponding to position 239; or K at the position corresponding to position 345, and G at the position corresponding to position 430.

3. The antigen binding molecule according to claim 1 or claim 2, wherein the Fc region comprises a polypeptide having: (i) C at the position corresponding to position 242, and C at the position corresponding to position 334, and (ii) A at the position corresponding to position 236, D at the position corresponding to position 239, E at the position corresponding to position 332, and L at the position corresponding to position 330.

4. The antigen binding molecule according to claim 1 or claim 2, wherein the Fc region comprises a polypeptide having: (i) C at the position corresponding to position 242, and C at the position corresponding to position 334, and (ii) A at the position corresponding to position 236, D at the position corresponding to position 239, and E at the position corresponding to position 332.

5. The antigen binding molecule according to claim 1 or claim 2, wherein the Fc region comprises a polypeptide having: (i) C at the position corresponding to position 242, and C at the position corresponding to position 334, and (ii) A at the position corresponding to position 236, and D at the position corresponding to position 239.

6. The antigen binding molecule according to claim 1 or claim 2, wherein the Fc region comprises a polypeptide having: (i) C at the position corresponding to position 242, and C at the position corresponding to position 334, and (ii) K at the position corresponding to position 345, and G at the position corresponding to position 430.

7. The antigen binding molecule according to any one of claims 1 to 6, wherein the Fc region comprises a polypeptide comprising an amino acid sequence having at least 60% sequence identity to SEQ ID NO:39, 38, 37, 41, 22, 21, 20 or 24.

8. A polypeptide, optionally isolated, comprising: an amino acid sequence having at least 60% sequence identity to SEQ ID NO:31 or 6, wherein the polypeptide comprises the following amino acid residues at the specified positions numbered relative to SEQ ID NO:31 or 6: (i) C at position 15, and C at position 107, and (ii) one or more of: A at position 9, D at position 12, L at position 103, E at position 105, K at position 118, and G at position 203.

9. The polypeptide according to claim 8, wherein the polypeptide comprises the following amino acid residues at the specified positions numbered relative to SEQ ID NO:31 or 6: (i) C at position 15, and C at position 107, and (ii) A at position 9, D at position 12, L at position 103, and E at position 105; or A at position 9, D at position 12, and E at position 105; or A at position 9, and D at position 12; or K at position 118, and G at position 203.

10. The polypeptide according to claim 8 or claim 9, wherein the polypeptide comprises the following amino acid residues at the specified positions numbered relative to SEQ ID NO:31 or 6: (i) C at position 15, and C at position 107, and (ii) A at position 9, D at position 12, L at position 103, and E at position 105.

11. The polypeptide according to claim 8 or claim 9, wherein the polypeptide comprises the following amino acid residues at the specified positions numbered relative to SEQ ID NO:31 or 6: (i) C at position 15, and C at position 107, and (ii) A at position 9, D at position 12, and E at position 105.

12. The polypeptide according to claim 8 or claim 9, wherein the polypeptide comprises the following amino acid residues at the specified positions numbered relative to SEQ ID NO:31 or 6: (i) C at position 15, and C at position 107, and (ii) A at position 9, and D at position 12.

13. The polypeptide according to claim 8 or claim 9, wherein the polypeptide comprises the following amino acid residues at the specified positions numbered relative to SEQ ID NO:31 or 6: (i) C at position 15, and C at position 107, and (ii) K at position 118, and G at position 203.

14. A polypeptide, optionally isolated, comprising the amino acid sequence of SEQ ID NO:39, 38, 37, 41, 22, 21, 20 or 24.

15. An Fc region, optionally isolated, comprising a polypeptide according to any one of claims 8 to 14.

16. An antigen-binding molecule, optionally isolated, comprising a polypeptide according to any one of claims 7 to 13, or an Fc region according to claim 14.

17. A nucleic acid, or a plurality of nucleic acids, optionally isolated, encoding an antigen-binding molecule according to any one of claim 1 to 7 or 16, a polypeptide according to any one of claims 8 to 14, or an Fc region according to claim 15.

18. An expression vector, or a plurality of expression vectors, comprising a nucleic acid or a plurality of nucleic acids according to claim 17.

19. A cell comprising an antigen-binding molecule according to any one of claim 1 to 7 or 16, a polypeptide according to any one of claims 8 to 14, an Fc region according to claim 15, a nucleic acid or a plurality of nucleic acids according to claim 17, or an expression vector or a plurality of expression vectors according to claim 18.

20. A method comprising culturing a cell comprising a nucleic acid or a plurality of nucleic acids according to claim 17, or an expression vector or a plurality of expression vectors according to claim 18, under conditions suitable for expression of the antigen-binding molecule, polypeptide or Fc region from the nucleic acid(s) or expression vector(s).

21. A composition comprising an antigen-binding molecule according to any one of claim 1 to 7 or 16, a polypeptide according to any one of claims 8 to 14, an Fc region according to claim 15, a nucleic acid or a plurality of nucleic acids according to claim 17, an expression vector or a plurality of expression vectors according to claim 18, or a cell according to claim 19.

22. An antigen-binding molecule according to any one of claim 1 to 7 or 16, a polypeptide according to any one of claims 8 to 14, an Fc region according to claim 15, a nucleic acid or a plurality of nucleic acids according to claim 17, an expression vector or a plurality of expression vectors according to claim 18, a cell according to claim 19, or a composition according to claim 21 for use in a method of medical treatment or prophylaxis.

23. An antigen-binding molecule according to any one of claim 1 to 7 or 16, a polypeptide according to any one of claims 8 to 14, an Fc region according to claim 15, a nucleic acid or a plurality of nucleic acids according to claim 17, an expression vector or a plurality of expression vectors according to claim 18, a cell according to claim 19, or a composition according to claim 21, for use in a method of treatment or prevention of a cancer, an infectious disease or an autoimmune disease.

24. A method, optionally an in vitro method, of killing cells expressing a target antigen, comprising contacting cells expressing the target antigen with an antigen-binding molecule according to any one of claim 1 to 7 or 16, a polypeptide according to any one of claims 8 to 14, an Fc region according to claim 15, a cell according to claim 19, or a composition according to claim 21.
